(19) United States
(12) Patent Application Publication
(10) Pub. No.: US 2018/0194856 A1
(43) Pub. Date: Jul. 12, 2018

(54) FUSION PROTEINS WHICH BIND TO HUMAN FC RECEPTORS

(71) Applicant: UCB Biopharma SPRL, Brussels (BE)

(72) Inventors: Robert Anthony GRIFFIN, Slough Berkshire (GB); David Paul HUMPHREYS, Slough Berkshire (GB); Shirley Jane PETERS, Slough Berkshire (GB)

(21) Appl. No.: 15/741,590

(22) PCT Filed: Jul. 6, 2016
(86) PCT No.: PCT/EP2016/065914

§ 371 Date: Jan. 3, 2018

(30) Foreign Application Priority Data
Jul. 6, 2015 (GB) 1511787.2

(51) Int. Cl. C07K 16/32; C07K 16/28; C07K 16/30; A61P 35/00

(52) U.S. Cl. CPC C07K 16/32; C07K 2317/52; C07K 16/30; A61P 35/00; C07K 2319/00; C07K 2317/56; C07K 2317/55; C07K 2317/622; C07K 2317/569; C07K 2317/53; C07K 2317/94; C07K 2317/14; C07K 2317/734; C07K 2317/24; C07K 16/2887

(57) ABSTRACT

The invention relates to fusion proteins which bind to human Fc-receptors. The fusion proteins are initially produced as monomers, and are capable of assembly into multimers at a target site of interest. The invention also relates to therapeutic compositions comprising the fusion proteins, and their widespread use in the treatment of diseases.

The invention relates to fusion proteins which bind to human Fc-receptors. The fusion proteins are initially produced as monomers, and are capable of assembly into multimers at a target site of interest. The invention also relates to therapeutic compositions comprising the fusion proteins, and their widespread use in the treatment of diseases.

### BACKGROUND

Monoclonal antibodies (mAb) represent one of the fastest growing sectors of medical treatment. They are now successfully employed in the treatment of many diseases, ranging from cancer to autoimmunity. These diseases are rapidly increasing in number due to our aging population and so new and more effective treatments are required. To maintain this growth in mAb-based treatments and increase their effectiveness we need to better understand the mechanisms of action of mAb and develop new technologies and formats that can augment even further their native therapeutic capabilities.

Almost all mAb depend on their Fc region for function which interfaces with key elements of the immune system; most notably complement and immune effector cells. Key to this interaction are the structural relationships between the mAb Fc region and the immune receptor molecules; the first component of complement (C1) and various Fc gamma Receptors (FcγRs). However, these relationships remain only partially understood.

It has recently been found that complement may be activated by IgG hexamers assembled at the cell surface. Specific noncovalent interactions between Fc segments of IgG antibodies were observed to result in the formation of ordered antibody hexamers after antigen binding on cells. The hexamers recruited and activated C1, triggering the complement cascade. (Diebolder C. A. et al., Science 343, 1260-1263, (2014).

Advances in antibody therapies have largely focussed on making the mAb more potent. In the case of enhanced effector functions this can often result in a less well tolerated drug with greater side-effects and dose-limiting toxicity. Thus, there remains a significant clinical need for improved antibody therapies with better safety profiles, which are highly effective and potent, and yet have fewer adverse side effects and low toxicity.

In the present invention we therefore provide improved fusion proteins which bind to human Fc-receptors. The fusion proteins are initially produced as monomers, and are capable of assembly into multimers at a target site of interest. The fusion proteins may be used to design a new class of antibody therapeutics, that are administered as relatively inert monomers, and assemble into highly potent multimers only when they reach a desired location at a target site of interest. The fusion proteins may thus provide improved therapeutic compositions with greater safety, which combine enhanced efficacy and potency with fewer adverse side effects and low toxicity.

### DESCRIPTION OF THE INVENTION

Unless defined otherwise, all technical and scientific terms used herein have the same meaning as commonly understood by one of skill in the art to which this invention belongs. All publications and patents referred to herein are incorporated by reference.

It will be appreciated that any of the embodiments described herein may be combined.

In the present specification the EU numbering system is used to refer to the residues in antibody domains, unless otherwise specified. This system was originally devised by Edelman et al, 1969 and is described in detail in Kabat et al, 1987. Edelman et al., 1969; “The covalent structure of an entire γG immunoglobulin molecule,” PNAS Biochemistry Vol.63 pp78-85. Kabat et al., 1987; in Sequences of Proteins of Immunological Interest, US Department of Health and Human Services, NIH, USA.

Where a position number and/or amino acid residue is given for a particular antibody isotype, it is intended to be applicable to the corresponding position and/or amino acid residue in any other antibody isotype, as is known by a person skilled in the art. When referring to an amino acid residue in a tailpiece derived from IgM or IgA, the position number given is the position number of the residue in naturally occurring IgM or IgA, according to conventional practice in the art.

The present invention provides a monomeric fusion protein comprising an antibody Fc-domain having two heavy chain Fc-regions derived from IgG;

wherein one or each heavy chain Fc-region is fused at its C-terminal to an antibody tailpiece;

wherein the tailpiece cysteine residue is modified to prevent disulphide bond formation.

In one embodiment, the tailpiece cysteine residue is deleted, substituted, or blocked with a thiol capping agent.

In one embodiment, the fusion protein of the present invention further comprises an antigen binding region. The antigen binding region may comprise any suitable antigen binding domain. In one example the antigen binding region may be derived from an antibody and may for example comprise a VH and/or a VL antigen binding domain. In one embodiment, the antigen binding region is selected from the group consisting of Fab, scFv, single domain antibody (dAb), and DARPin. In one example a single domain antibody may be a VH, VL or VHH domain. In one embodiment, the antigen binding region is fused to the N-terminus of the heavy chain Fc-region. The antigen binding region may be fused directly to the N-terminus of the heavy chain Fc-region. Alternatively it may be fused indirectly by means of an intervening amino acid sequence. For example, a short peptide linker or a hinge sequence may be provided between the fusion partner and the heavy chain Fc-region.

In one embodiment, the fusion protein of the present invention further comprises a fusion partner. Typically the term ‘fusion partner’ refers to an antigen, pathogen-associated molecular pattern (PAMP), drug, ligand, receptor, cytokine or chemokine. In one example the ‘fusion partner’ does not include an antibody or a variable domain derived from an antibody. In one embodiment, the fusion partner is fused to the N-terminus of the heavy chain Fc-region. The fusion partner may be fused directly to the N-terminus of the heavy chain Fc-region. Alternatively it may be fused indirectly by means of an intervening amino acid sequence. For example, a short peptide linker or a hinge sequence may be provided between the fusion partner and the heavy chain Fc-region.

The antibody Fc-domain component of the monomers of the present invention may be derived from any suitable species. In one embodiment the antibody Fc-domain is derived from a human Fc-domain.

The antibody Fc-domain may be derived from any suitable class of antibody, including IgA (including subclasses IgA1 and IgA2), IgD, IgE, IgG (including subclasses IgG1, IgG2, IgG3 and IgG4), and IgM. Typically, the antibody Fc-domain is derived from IgG. In one embodiment the antibody Fc-domain is derived from IgG1. In one embodiment the antibody Fc-domain is derived from IgG2. In one embodiment the antibody Fc domain is derived from IgG3. In one embodiment the antibody Fc domain is derived from IgG4.

The antibody Fc-domain comprises two individual polypeptide chains, each referred to as a heavy chain Fc-region. The two heavy chain Fc-regions dimerise to create the antibody Fc-domain. Whilst the two heavy chain Fc-regions that together form the antibody Fc domain may be different from one another it will be appreciated that these will usually be the same as one another.

Typically each heavy chain Fc-region comprises or consists of two or three heavy chain constant domains.

In native antibodies, the heavy chain Fc-region of IgA, IgD and IgG is composed of two heavy chain constant domains (CH2 and CH3) and that of IgE and IgM is composed of three heavy chain constant domains (CH2, CH3 and CH4). These dimerise to create the antibody Fc domain.

In the present invention, the heavy chain Fc-region may comprise heavy chain constant domains from one or more different classes of antibody, for example one, two or three different classes.

In one embodiment the heavy chain Fc-region comprises CH2 and CH3 domains derived from IgG1.

In one embodiment the heavy chain Fc-region comprises CH2 and CH3 domains derived from IgG2.

In one embodiment the heavy chain Fc-region comprises CH2 and CH3 domains derived from IgG3.

In one embodiment the heavy chain Fc-region comprises CH2 and CH3 domains derived from IgG4.

In our earlier application, PCT/EP2015/054687, we disclose that the CH3 domain plays a significant role in the polymerisation of fusion proteins comprising an antibody Fc-domain and a tail-piece sequence. The amino acid at position 355 of the CH3 domain was found to have a particularly strong effect.

Thus in one embodiment, the heavy chain Fc-region comprises a CH3 domain derived from IgG1.

In one embodiment, the heavy chain Fc-region comprises a CH2 domain derived from IgG4 and a CH3 domain derived from IgG1.

In one embodiment, the heavy chain Fc-region comprises an arginine residue at position 355.

In one embodiment the heavy chain Fc-region comprises a CH4 domain from IgM. The IgM CH4 domain is typically located between the CH3 domain and the tailpiece.

In one embodiment the heavy chain Fc-region comprises CH2 and CH3 domains derived from IgG and a CH4 domain derived from IgM.

It will be appreciated that the heavy chain constant domains for use in producing a heavy chain Fc-region of the present invention may include variants of the naturally occurring constant domains described above. Such variants may comprise one or more amino acid variations compared to wild type constant domains. In one example the heavy chain Fc-region of the present invention comprises at least one constant domain which varies in sequence from the wild type constant domain. It will be appreciated that the variant constant domains may be longer or shorter than the wild type constant domain. Preferably the variant constant domains are at least 50% identical or similar to a wild type constant domain. The term “identity”, as used herein, indicates that at any particular position in the aligned sequences, the amino acid residue is identical between the sequences. The term “similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains).

Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). In one example the variant constant domains are at least 60% identical or similar to a wild type constant domain. In another example the variant constant domains are at least 70% identical or similar. In another example the variant constant domains are at least 80% identical or similar. In another example the variant constant domains are at least 90% identical or similar. In another example the variant constant domains are at least 95% identical or similar.

In the fusion proteins of the invention, one or each heavy chain Fc-region is fused at its C-terminus to an antibody tailpiece, wherein the tailpiece cysteine residue is modified to prevent disulphide bond formation.

The antibody tailpiece of the invention may be derived from any suitable species. Antibody tailpieces are evolutionarily conserved and are found in most species, including primitive species such as teleosts. In one embodiment, the antibody tailpiece is derived from a human antibody.

In humans, IgM and IgA occur naturally as covalent multimers of the common H₂L₂ antibody unit. IgM occurs as a pentamer when it has incorporated a J-chain, or as a hexamer when it lacks a J-chain. IgA occurs as monomer and dimer forms. The heavy chains of IgM and IgA possess an 18 amino acid extension to the C-terminal constant domain, known as a tailpiece. This tailpiece naturally includes a cysteine residue that forms a disulphide bond with adjacent heavy chains in the polymer, and is believed to have an important role in polymerisation. The tailpiece also contains a glycosylation site.

In our earlier application, PCT/EP2015/054687, we disclosed that recombinant fusion proteins comprising an antibody Fc-domain derived from IgG and a tailpiece, are synthesised predominantly as hexamers.

The present inventors have unexpectedly found that modification of the tailpiece cysteine residue results in fusion proteins with very unusual multimerisation properties. The fusion proteins of the present invention are synthesized predominantly as monomers, and are capable of assembly into multimers at high concentrations, as shown in FIG. 1. The fusion proteins may be used to design a new class of antibody therapeutics, that are administered as relatively inert monomers, and assemble into highly potent multimers only when they reach a desired location at a target site of interest. The fusion proteins may thus provide improved therapeutic compositions with greater safety, which combine enhanced efficacy and potency with fewer adverse side effects and low toxicity.

In one example the monomers of the present invention are directed to a target site of interest via the Fc domain and/or, where present, the antigen binding region and/or fusion partner. For example, the monomer of the present invention may comprise an antigen binding region which may bind an antigen expressed on the surface of a cell, such as a tumor cell or an immune cell, such that assembly of the monomer into a highly potent multimer may occur on the tumor cell or immune cell surface. Examples of suitable antigens include HER2/neu or CD20.

In the fusion proteins of the present invention, the tailpiece cysteine residue is modified to prevent disulphide bond formation. The cysteine residue may be modified using any any suitable method that prevents disulphide bond formation.

In one embodiment, the tailpiece cysteine residue is mutated. In one embodiment, the tailpiece cysteine residue is deleted. In one embodiment, the tailpiece cysteine residue is substituted with another amino acid residue.

In one embodiment, the antibody tailpiece is derived from human IgM or IgA, wherein the tailpiece cysteine residue normally found at position 575 of IgM or position 495 of IgA has been deleted or substituted with another amino acid residue.

In one embodiment, the tailpiece cysteine residue normally found at position 575 of IgM is substituted with a serine, threonine or alanine residue (C575S, C575T, or C575A).

In one embodiment, the tailpiece cysteine residue normally found at position 495 of IgA is substituted with a serine, threonine or alanine residue (C495S, C495T, or C495A).

In one embodiment, the tailpiece cysteine residue is blocked with a thiol capping agent. A thiol capping agent is a compound that reacts with sulphydryl groups in reduced cysteine residues, preventing them from forming disulphide bonds. Examples of suitable thiol capping agents include N-ethylmaleimide, iodoacetic acid, or iodoacetamide.

In one embodiment, the thiol capping agent used to block the tailpiece cysteine residue is conjugated to a drug. The capping reaction thus effectively forms a bridge between the drug and the cysteine residue in the target protein, conjugating the drug to the tailpiece.

In one embodiment, the tailpiece comprises all or part of an 18 amino acid tailpiece sequence from human IgM or IgA as shown in Table 1, wherein the cysteine residue normally found at position 575 of IgM, or position 495 of IgA, is deleted, substituted, or blocked with a thiol capping agent.

The tailpiece may be fused directly to the C-terminus of the heavy chain Fc-region. Alternatively, it may be fused indirectly by means of an intervening amino acid sequence. For example, a short linker sequence may be provided between the tailpiece and the heavy chain Fc-region.

The tailpiece of the present invention may include variants or fragments of the tailpiece sequences described above. A variant of an IgM or IgA tailpiece typically has an amino acid sequence which is identical to the described sequence in 8, 9, 10, 11, 12, 13, 14, 15, 16, or 17 of the 18 amino acid positions shown in Table 1. A fragment typically comprises 8, 9, 10, 11, 12, 13, 14, 15, 16, or 17 amino acids. The tailpiece may be a hybrid IgM/IgA tailpiece. Fragments of variants are also envisaged.

TABLE 1
Tailpiece sequences

| Tailpiece | Sequence |
|---|---|
| IgM | PTLYNVSLVMSDTAGTCY SEQ ID NO: 1 |
| IgA | PTHVNVSVVMAEVDGTCY SEQ ID NO: 2 |

Each heavy chain Fc-region of the present invention may optionally possess a native or a modified hinge region at its N-terminus.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama or goat hinge regions. Other modified hinge regions may comprise a complete hinge region derived from an antibody of a different class or subclass from that of the heavy chain Fc-region. Alternatively, the modified hinge region may comprise part of a natural hinge or a repeating unit in which each unit in the repeat is derived from a natural hinge region. In a further alternative, the natural hinge region may be altered by converting one or more cysteine or other residues into neutral residues, such as serine or alanine, or by converting suitably placed residues into cysteine residues. By such means the number of cysteine residues in the hinge region may be increased or decreased. Other modified hinge regions may be entirely synthetic and may be designed to possess desired properties such as length, cysteine composition and flexibility.

A number of modified hinge regions have already been described for example, in U.S. Pat. No. 5,677,425, WO9915549, WO2005003170, WO2005003169, WO2005003170, WO9825971 and WO2005003171 and these are incorporated herein by reference.

Examples of suitable hinge sequences are shown in Table 2.

In one embodiment, the heavy chain Fc-region possesses an intact hinge region at its N-terminus.

In one embodiment the heavy chain Fc-region and hinge region are derived from IgG4 and the hinge region comprises the mutated sequence CPPC (SEQ ID NO: 11). The core hinge region of human IgG4 naturally contains the sequence CPSC (SEQ ID NO: 12), compared to IgG1 which contains the sequence CPPC. The serine residue present in the IgG4 sequence leads to increased flexibility in this region, and therefore a proportion of molecules form disulphide bonds within the same protein chain (an intrachain disulphide) rather than bridging to the other heavy chain in the IgG molecule to form the interchain disulphide. (Angal S. et al, Mol Immunol, Vol 30(1), p105-108, 1993). Changing the serine residue to proline to give the same core sequence as IgG1 allows complete formation of inter-chain disulphides in the IgG4 hinge region, thus reducing heterogeneity in the purified product. This altered isotype is termed IgG4P.

TABLE 2
Hinge sequences

| Hinge | Sequence |
|---|---|
| Human IgA1 | VPSTPPTPSPSTPPTPSPS SEQ ID NO: 3 |
| Human IgA2 | VPPPPP SEQ ID NO: 4 |
| Human IgD | ESPKAQASSVPTAQPQAEGSLAKATTAPATTR |
| | NTGRGGEEKKKEKEKEEQEERETKTP SEQ ID |
| | NO: 5 |
| Human IgG1 | EPKSCDKTHTCPPCP SEQ ID NO: 6 |
| Human IgG2 | ERKCCVECPPCP SEQ ID NO: 7 |
| Human IgG3 | ELKTPLGDTTHTCPRCPEPKSCDTPPPCPRCPE |
| | PKSCDTPPPCPRCPEPKSCDTPPPCPRCP SEQ |
| | ID NO: 8 |
| Human IgG4 | ESKYGPPCPSCP SEQ ID NO: 9 |
| Human IgG4(P) | ESKYGPPCPPCP SEQ ID NO: 10 |
| Recombinant v1 | CPPC SEQ ID NO: 11 |
| Recombinant v2 | CPSC SEQ ID NO: 12 |
| Recombinant v3 | CPRC SEQ ID NO: 13 |
| Recombinant v4 | SPPC SEQ ID NO: 14 |
| Recombinant v5 | CPPS SEQ ID NO: 15 |
| Recombinant v6 | SPPS SEQ ID NO: 16 |
| Recombinant v7 | DKTHTCAA SEQ ID NO: 17 |
| Recombinant v8 | DKTHTCPPCPA SEQ ID NO: 18 |
| Recombinant v9 | DKTHTCPPCPATCPPCPA SEQ ID NO: 19 |
| Recombinant v10 | DKTHTCPPCPATCPPCPATCPPCPA SEQ ID |
| | NO: 20 |
| Recombinant v11 | DKTHTCPPCPAGKPTLYNSLVMSDTAGTCY |
| | SEQ ID NO: 21 |
| Recombinant v12 | DKTHTCPPCPAGKPTHVNVSVVMAEVDGTCY |
| | SEQ ID NO: 22 |
| Recombinant v13 | DKTHTCCVECPPCPA SEQ ID NO: 23 |
| Recombinant v14 | DKTHTCPRCPEPKSCDTPPPCPRCPA SEQ |
| | ID NO: 24 |
| Recombinant v15 | DKTHTCPSCPA SEQ ID NO: 25 |

In one embodiment, a monomeric fusion protein of the invention comprises an amino acid sequence as provided in FIG. 2, optionally with an alternative hinge or tailpiece sequence.

Accordingly in one example the present invention provides a monomeric fusion protein comprising two identical polypeptide chains, each polypeptide chain comprising or consisting of the sequence given in any one of SEQ ID NOs 26 to 37.

In one example where the hinge may be varied from the sequences given in SEQ ID NOs 26 to 37, the present invention provides a monomeric fusion protein comprising two identical polypeptide chains, each polypeptide chain comprising or consisting of the sequence given in amino acids 6 to 222 of any one of SEQ ID NOs 26 to 29, or the sequence given in amino acids 6 to 333 of SEQ ID NOs 30 or 31, or the sequence given in amino acids 6 to 221 of any one of SEQ ID NOs 32 to 35, or the sequence given in amino acids 6 to 332 of SEQ ID NOs 36 or 37.

In one embodiment, a monomeric fusion protein of the invention comprises an amino acid sequence as provided in FIG. 3. Accordingly in one example the present invention provides a monomeric fusion protein comprising or consisting of an amino acid sequence given in any one of SEQ ID Nos 38 to 69.

The monomeric fusion protein of the invention is capable of concentration-dependent assembly into multimers. The multimer may comprise two, three, four, five, six, seven, eight, nine, ten, eleven or twelve or more monomer units. Such a multimer may alternatively be referred to as a dimer, trimer, tetramer, pentamer, hexamer, heptamer, octamer, nonamer, decamer, undecamer, dodecamer, etc., respectively. In one example, the monomeric fusion protein assembles into a hexamer.

Thus in one embodiment, the present invention provides a mixture comprising a monomeric fusion protein of the invention and a multimer, said multimer comprising two or more monomer units.

In one embodiment, the mixture comprises a monomeric fusion protein of the invention and a hexamer.

In one embodiment, the mixture comprises greater than 55% monomer, for example, greater than 65%, greater than 75%, greater than 85%, greater than 90% or greater than 95% monomer.

In one embodiment, the mixture is enriched for the monomeric form of the fusion protein of the invention. In one example, the term “enriched” means that greater than 80% of the fusion protein of the invention is present in the mixture in monomeric form, such as greater than 90% or greater than 95%. It will be appreciated that the proportion of monomer in a sample can be determined using any suitable method such as analytical size exclusion chromatography, as described herein below.

In one embodiment, the monomeric fusion protein of the invention is produced or formulated using a component that stabilises the monomeric form of the protein. The component may increase the ratio of monomer to multimer in a mixture, so that a higher proportion of the protein is present in monomeric form than would otherwise be the case. Examples of suitable components include pharmaceutical excipients, diluents or carriers which enable the monomeric fusion protein of the invention to be formulated at high concentration prior to administration.

The monomeric fusion proteins of the present invention may comprise one or more mutations that alter the functional properties of the proteins, for example, binding to Fc-receptors such as leukocyte receptors, complement or FcRn. Examples of mutations that alter the functional properties of the proteins are described in detail in our earlier application, PCT/EP2015/054687. It will be appreciated that any of these mutations may be combined in any suitable manner to achieve the desired functional properties, and/or combined with other mutations to alter the functional properties of the proteins.

The present invention also provides an isolated DNA sequence encoding a polypeptide chain of the present invention, or a component part thereof. The DNA sequence may comprise synthetic DNA, for instance produced by chemical processing, cDNA, genomic DNA or any combination thereof.

DNA sequences which encode a polypeptide chain of the present invention can be obtained by methods well known to those skilled in the art. For example, DNA sequences coding for part or all of a polypeptide chain may be synthesised as desired from the determined DNA sequences or on the basis of the corresponding amino acid sequences.

In one example, a monomeric fusion protein of the invention is encoded by a DNA sequence as provided in FIG. 3. Accordingly in one example the present invention provides a DNA sequence given in any one of SEQ ID Nos 38 to 69.

Standard techniques of molecular biology may be used to prepare DNA sequences coding for a polypeptide chain of the present invention. Desired DNA sequences may be synthesised completely or in part using oligonucleotide synthesis techniques. Site-directed mutagenesis and polymerase chain reaction (PCR) techniques may be used as appropriate.

The present invention also relates to a cloning or expression vector comprising one or more DNA sequences of the present invention. Accordingly, provided is a cloning or expression vector comprising one or more DNA sequences encoding a polypeptide chain of the present invention, or a component part thereof.

General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing.

Also provided is a host cell comprising one or more cloning or expression vectors comprising one or more DNA sequences encoding a monomeric fusion protein of the present invention. Any suitable host cell/vector system may be used for expression of the DNA sequences encoding the monomeric fusion protein of the present invention. Bacterial, for example E. coli, and other microbial systems such as Saccharomyces or Pichia may be used or eukaryotic, for example mammalian, host cell expression systems may also be used. Suitable mammalian host cells include CHO cells. Suitable types of chinese hamster ovary (CHO cells) for use in the present invention may include CHO and CHO-K1 cells, including dhfr-CHO cells, such as CHO-DG44 cells and CHO-DXB11 cells, which may be used with a DHFR selectable marker, or CHOK1-SV cells which may be used with a glutamine synthetase selectable marker. Other suitable host cells include NSO cells.

The present invention also provides a process for the production of a monomeric fusion protein according to the present invention, comprising culturing a host cell containing a vector of the present invention under conditions suitable for expression of the monomeric fusion protein, and isolating and optionally purifying the monomeric fusion protein.

The monomeric fusion proteins of the present invention are expressed at good levels from host cells. Thus the properties of the monomeric fusion protein are conducive to commercial processing.

The monomeric fusion proteins of the present invention may be made using any suitable method. In one embodiment, the monomeric fusion protein of the invention may be produced under conditions which minimise aggregation. In one example, aggregation may be minimised by the addition of preservative to the culture media, culture supernatant, or purification media. Examples of suitable preservatives include ethylenediaminetetraacetic acid (EDTA), ethyleneglycoltetraacetic acid (EGTA), or acidification to below pH 6.0.

In one embodiment there is provided a process for purifying a monomeric fusion protein of the present invention comprising the steps: performing anion exchange chromatography in non-binding mode such that the impurities are retained on the column and the monomeric fusion protein is eluted.

In one embodiment the purification employs affinity capture on an FcRn, FcγR or C-reactive protein column.

In one embodiment the purification employs protein A.

Suitable ion exchange resins for use in the process include Q.FF resin (supplied by GE-Healthcare). The step may, for example be performed at a pH about 8. The process may further comprise an initial capture step employing cation exchange chromatography, performed for example at a pH of about 4 to 5, such as 4.5. The cation exchange chromatography may, for example employ a resin such as CaptoS resin or SP sepharose FF (supplied by GE-Healthcare). The monomeric fusion protein can then be eluted from the resin with an ionic salt solution such as sodium chloride, for example at a concentration of 200 mM.

The chromatography step or steps may include one or more washing steps, as appropriate.

The purification process may also comprise one or more filtration steps, such as a diafiltration step.

The monomeric fusion proteins of the invention can be purified according to molecular size, for example by size exclusion chromatography.

Thus in one embodiment there is provided a purified monomeric fusion protein according to the invention, in substantially purified from, in particular free or substantially free of endotoxin and/or host cell protein or DNA.

Purified form as used herein is intended to refer to at least 90% purity, such as 91, 92, 93, 94, 95, 96, 97, 98, 99% w/w or more pure.

Substantially free of endotoxin is generally intended to refer to an endotoxin content of 1 EU per mg protein product or less, such as 0.5 or 0.1 EU per mg product.

Substantially free of host cell protein or DNA is generally intended to refer to a host cell protein and/or DNA content of 400 μg per mg of protein product or less, such as 100 μg per mg product or less, in particular 20 μg per mg product.

As the monomeric fusion proteins of the present invention are useful in the treatment and/or prophylaxis of a pathological condition, the present invention also provides a pharmaceutical or diagnostic composition comprising a monomeric fusion protein of the present invention in combination with one or more of a pharmaceutically acceptable excipient, diluent or carrier.

The present invention also provides a process for preparation of a pharmaceutical or diagnostic composition comprising adding and mixing the monomeric fusion protein of the present invention together with one or more of a pharmaceutically acceptable excipient, diluent or carrier.

In one embodiment, the pharmaceutical composition comprises a component that stabilises the monomeric form of the protein or increases the ratio of monomer to multimer in a mixture.

The monomeric fusion protein may be the sole active ingredient in the pharmaceutical or diagnostic composition, or may be accompanied by other active ingredients including antibody ingredients or non-antibody ingredients such as other drug molecules.

The pharmaceutical compositions suitably comprise a therapeutically effective amount of the monomeric fusion protein of the invention. The term “therapeutically effective amount” as used herein refers to an amount of a therapeutic agent needed to treat, ameliorate or prevent a targeted disease or condition, or to exhibit a detectable therapeutic or preventative effect. For any medicine, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. This amount can be determined by routine experimentation and is within the judgement of the clinician. Generally, a therapeutically effective amount will be from 0.01 mg/kg to 500 mg/kg, for example 0.1 mg/kg to 200 mg/kg, such as 100 mg/kg. Pharmaceutical compositions may be conveniently presented in unit dose forms containing a predetermined amount of a monomeric fusion protein of the invention per dose.

Compositions may be administered individually to a patient or may be administered in combination (e.g. simultaneously, sequentially or separately) with other agents, drugs or hormones.

The dose at which the monomeric fusion protein of the present invention is administered depends on the nature of the condition to be treated, the extent of the disease present, and on whether the monomeric fusion protein is being used prophylactically or to treat an existing condition.

The frequency of dose will depend on the half-life of the monomeric fusion protein and the duration of its effect. If the monomeric fusion protein has a short half-life (e.g. 2 to 10 hours) it may be necessary to give one or more doses per day. Alternatively, if the monomeric fusion protein has a long half-life (e.g. 2 to 15 days), and/or long lasting pharmacodynamic effects, it may only be necessary to give a dosage once per day, once per week or even once every 1 or 2 months.

In one embodiment the dose is delivered bi-weekly, i.e. twice a month.

Half-life as employed herein is intended to refer to the duration of the monomeric fusion protein in circulation, for example in serum or plasma.

Pharmacodynamics as employed herein refers to the profile and, in particular, duration of the biological action of the monomeric fusion protein.

The pharmaceutically acceptable carrier should not itself induce the production of antibodies harmful to the individual receiving the composition and should not be toxic. Suitable carriers may be large, slowly metabolised macromolecules such as proteins, polypeptides, liposomes, polysaccharides, polylactic acids, polyglycolic acids, polymeric amino acids, amino acid copolymers and inactive virus particles. Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in therapeutic compositions may additionally contain liquids such as water, saline, glycerol and ethanol. Additionally, auxiliary substances, such as wetting or emulsifying agents or pH buffering substances, may be present in such compositions.

A thorough discussion of pharmaceutically acceptable carriers is available in Remington's Pharmaceutical Sciences (Mack Publishing Company, N.J. 1991).

The therapeutic suspensions or solution formulations can also contain one or more excipients. Excipients are well known in the art and include buffers (e.g., citrate buffer, phosphate buffer, acetate buffer and bicarbonate buffer), amino acids, urea, alcohols, ascorbic acid, phospholipids, proteins (e.g., serum albumin), EDTA, sodium chloride, liposomes, mannitol, sorbitol, and glycerol. Solutions or suspensions can be encapsulated in liposomes or biodegradable microspheres.

The formulation will generally be provided in a substantially sterile form employing sterile manufacture processes. This may include production and sterilization by filtration of the buffered solvent/solution used for the formulation, aseptic suspension of the protein in the sterile buffered solvent solution, and dispensing of the formulation into sterile receptacles by methods familiar to those of ordinary skill in the art.

Suitable forms for administration include forms suitable for parenteral administration, e.g. by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilising and/or dispersing agents. The monomeric fusion protein may be in the form of nanoparticles. Alternatively, the monomeric fusion protein may be in dry form, for reconstitution before use with an appropriate sterile liquid.

Once formulated, the compositions of the invention can be administered directly to the subject. The subjects to be treated can be animals. However, in one or more embodiments the compositions are adapted for administration to human subjects.

The pharmaceutical compositions of this invention may be administered by any number of routes including, but not limited to, oral, intravenous, intramuscular, intra-arterial, intramedullary, intrathecal, intraventricular, transdermal, transcutaneous (for example, see WO98/20734), subcutaneous, intraperitoneal, intranasal, enteral, topical, sublingual, intravaginal or rectal routes. Typically, the therapeutic compositions may be prepared as injectables, either as liquid solutions or suspensions. Solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule

It will be appreciated that the active ingredient in the composition will be a protein molecule. As such, it will be susceptible to degradation in the gastrointestinal tract. Thus, if the composition is to be administered by a route using the gastrointestinal tract, the composition will need to contain agents which protect the protein from degradation but which release the protein once it has been absorbed from the gastrointestinal tract.

In one embodiment the formulation is provided as a formulation for topical administrations including inhalation.

In one embodiment we provide the monomeric fusion protein of the invention for use in therapy. In one embodiment we provide the use of a monomeric fusion protein of the invention for the manufacture of a medicament.

In one embodiment we provide the monomeric fusion protein of the invention for use in the treatment of cancer.

In one embodiment we provide the use of a monomeric fusion protein of the invention for the manufacture of a medicament for the treatment of cancer.

Examples of cancers which may be treated using the monomeric fusion protein of the invention include colorectal cancer, liver cancer, prostate cancer, pancreatic cancer, breast cancer, ovarian cancer, thyroid cancer, renal cancer, bladder cancer, head and neck cancer or lung cancer. In one embodiment the cancer is skin cancer, such as melanoma. In one embodiment the cancer is Leukemia. In one embodiment the cancer is glioblastoma, medulloblastoma or neuroblastoma. In one embodiment the cancer is a neuroendocrine cancer. In one embodiment the cancer is Hodgkin's or non-Hodgkins lymphoma.

In one embodiment we provide the monomeric fusion protein of the invention for use in the treatment of immune disorders.

In one embodiment we provide the use of the monomeric fusion protein of the invention for the preparation of a medicament for the treatment of immune disorders.

Examples of immune disorders which may be treated using the monomeric fusion protein of the invention include autoimmune diseases, for example Acute Disseminated Encephalomyelitis (ADEM), Acute necrotizing hemorrhagic leukoencephalitis, Addison's disease, Agammaglobulinemia, Alopecia areata, Amyloidosis, ANCA-associated vasculitis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency , Autoimmune inner ear disease (AIED), Autoimmune myocarditis, Autoimmune pancreatitis, Autoimmune retinopathy, Autoimmune thrombocytopenic purpura (ATP), Autoimmune thyroid disease, Autoimmune urticarial, Axonal & nal neuropathies, Balo disease, Behcet's disease, Bullous pemphigoid, Cardiomyopathy, Castleman disease, Celiac disease, Chagas disease, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Dilated cardiomyopathy, Discoid lupus, Dressler's syndrome, Endometriosis, Eosinophilic angiocentric fibrosis, Eosinophilic fasciitis, Erythema nodosum, Experimental allergic encephalomyelitis, Evans syndrome, Fibrosing alveolitis, Giant cell arteritis (temporal arteritis), Glomerulonephritis, Goodpasture's syndrome, Granulomatosis with Polyangiitis (GPA) see Wegener's, Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic hypocomplementemic tubulointestitial nephritis, Idiopathic thrombocytopenic purpura (ITP), IgA nephropathy, IgG4-related disease, IgG4-related sclerosing disease, Immunoregulatory lipoproteins, Inflammatory aortic aneurysm, Inflammatory pseudotumour, Inclusion body myositis, Insulin-dependent diabetes (type1), Interstitial cystitis, Juvenile arthritis, Juvenile diabetes, Kawasaki syndrome, Kuttner's tumour, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Mediastinal fibrosis, Meniere's disease, Microscopic polyangiitis, Mikulicz's syndrome, Mixed connective tissue disease (MCTD), Mooren's ulcer, Mucha-Habermann disease, Multifocal fibrosclerosis, Multiple sclerosis, Myasthenia gravis, Myositis, Narcolepsy, Neuromyelitis optica (Devic's), Neutropenia, Ocular cicatricial pemphigoid, Optic neuritis, Ormond's disease (retroperitoneal fibrosis), Palindromic rheumatism, PANDAS (Pediatric Autoimmune Neuropsychiatric Disorders Associated with Streptococcus), Paraneoplastic cerebellar degeneration, Paraproteinemic polyneuropathies, Paroxysmal nocturnal hemoglobinuria (PNH), Parry Romberg syndrome, Parsonnage-Turner syndrome, Pars planitis (peripheral uveitis), Pemphigus vulgaris, Periaortitis, Periarteritis, Peripheral neuropathy, Perivenous encephalomyelitis, Pernicious anemia, POEMS syndrome, Polyarteritis nodosa, Type I, II, & Ill autoimmune polyglandular syndromes, Polymyalgia rheumatic, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reflex sympathetic dystrophy, Reiter's syndrome, Relapsing polychondritis, Restless legs syndrome, Retroperitoneal fibrosis (Ormond's disease), Rheumatic fever, Rheumatoid arthritis, Riedel's thyroiditis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombotic, thrombocytopenic purpura (TTP), Tolosa-Hunt syndrome, Transverse myelitis, Ulcerative colitis, Undifferentiated connective tissue disease (UCTD), Uveitis, Vasculitis, Vesiculobullous dermatosis, Vitiligo, Waldenstrom Macroglobulinaemia, Warm idiopathic haemolytic anaemia and Wegener's granulomatosis (now termed Granulomatosis with Polyangiitis (GPA).

In one embodiment the autoimmune disease is selected from immune thrombocytopenia (ITP), chronic inflammatory demyelinating polyneuropathy (CIDP), Kawasaki disease and Guillain-Barre syndrome (GBS).

In one embodiment the monomeric fusion proteins of the invention are employed in the treatment or prophylaxis of epilepsy or seizures.

In one embodiment the monomeric fusion proteins and fragments according to the disclosure are employed in the treatment or prophylaxis of multiple sclerosis.

The monomeric fusion protein according to the present disclosure may be employed in treatment or prophylaxis.

The monomeric fusion protein of the present invention may also be used in diagnosis, for example in the in vivo diagnosis and imaging of disease states involving Fc-receptors, such as B-cell related lymphomas.

### FIGURE LEGENDS

FIG. 1

1(a) Fusion proteins in which the tailpiece cysteine residue has been deleted or substituted with another amino acid residue are expressed predominantly as monomers and are capable of concentration-dependent assembly into hexamers.

1(b) Control proteins lacking the modified tailpiece are unable to assemble into hexamers, even at the highest concentrations tested.

1(c) Control proteins comprising an unmodified tailpiece with an intact cysteine residue are expressed predominantly in hexameric form.

FIG. 2

Example amino acid sequences of a polypeptide chain of a monomeric fusion protein. In each sequence, the tailpiece sequence is underlined, and any mutations are shown in bold and underlined. The hinge is in bold. In constructs comprising a CH4 domain from IgM, this region is shown in italics.

FIG. 3

Example amino acid and DNA sequences for antibody fusion proteins of the invention. The tailpiece sequence is underlined.

FIG. 4

Chromatographs demonstrating that full length antibody fusion proteins comprising the modified tailpiece of the invention exhibit concentration-dependent multimerisation into a high molecular weight species (HMWS).

4(a) Rituximab IgG1 FL IgM tp C575S

4(b) Trastuzumab IgG1 FL IgM tp C575S

FIG. 5

Complement killing of cells by a fusion protein of the invention. The graphs show complement killing of CD20 positive Raji cells and CD20 positive Ramos cells. The results demonstrate that the anti-CD20 antibody rituximab shows enhanced CDC when modified with the tailpiece of the invention.

### EXAMPLES

### Example 1

### Molecular Biology

DNA sequences were assembled using standard molecular biology methods, including PCR, restriction-ligation cloning, point mutagenesis (Quikchange) and Sanger sequencing. Expression constructs were cloned into expression plasmids (pNAFL, pNAFH) suitable for both transient and stable expression in CHO cells. Other examples of suitable expression vectors include pCDNA3 (Invitrogen).

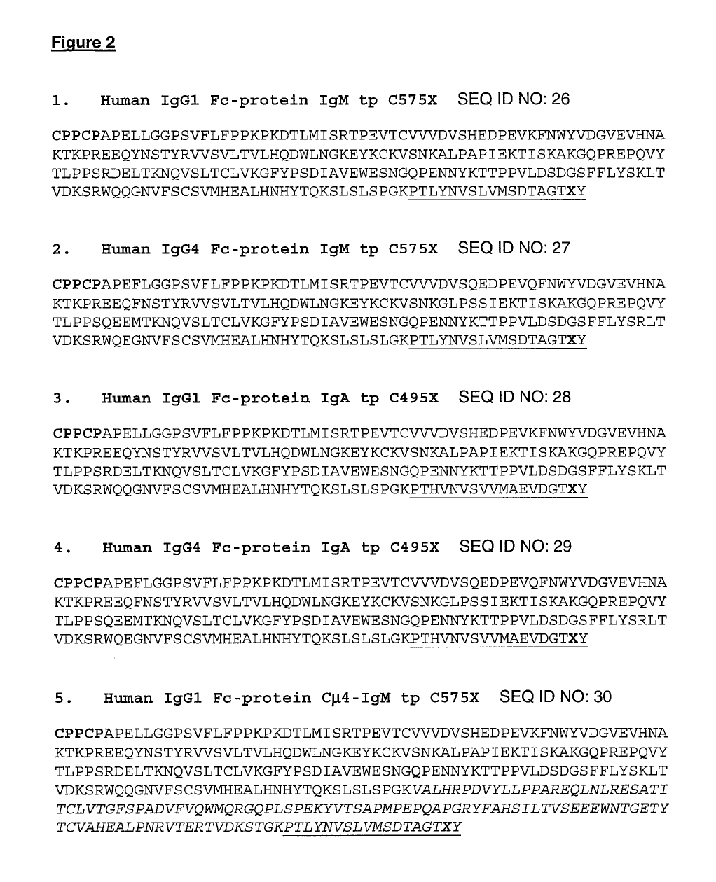

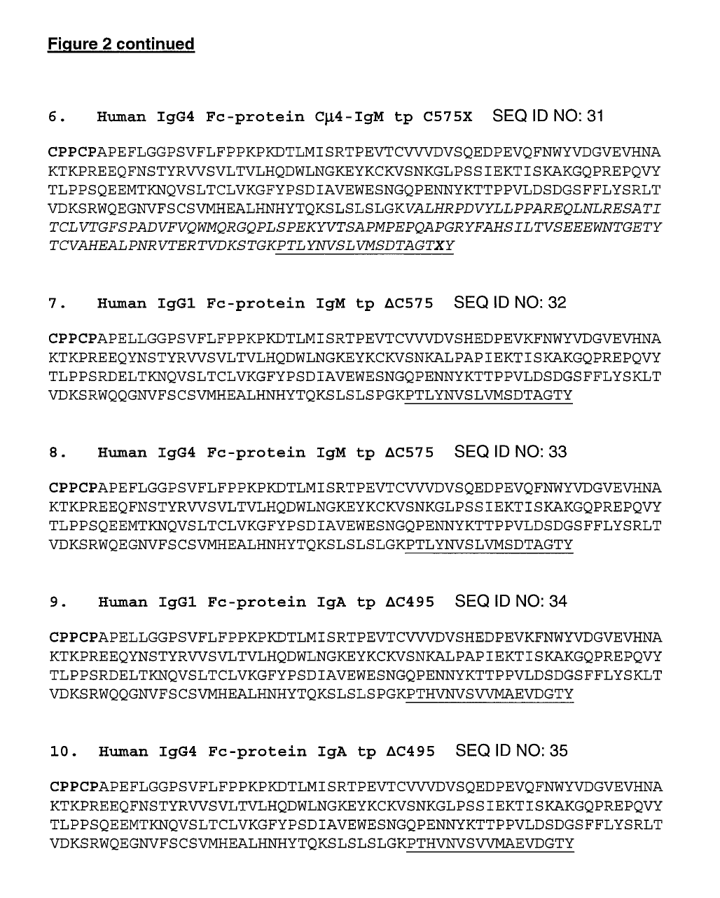

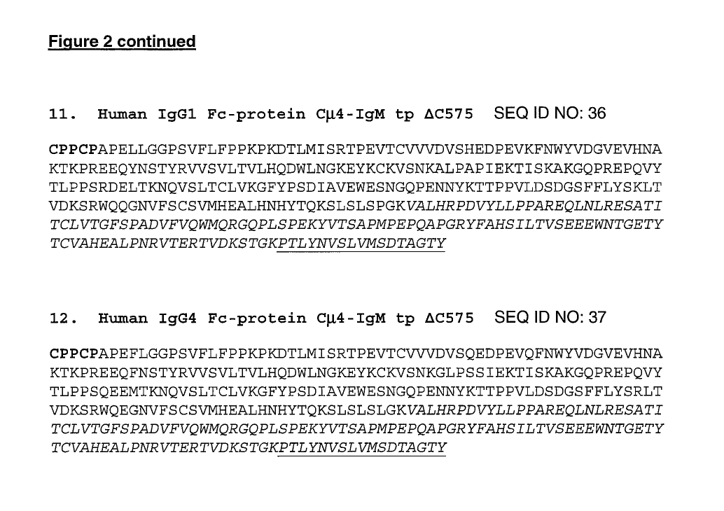

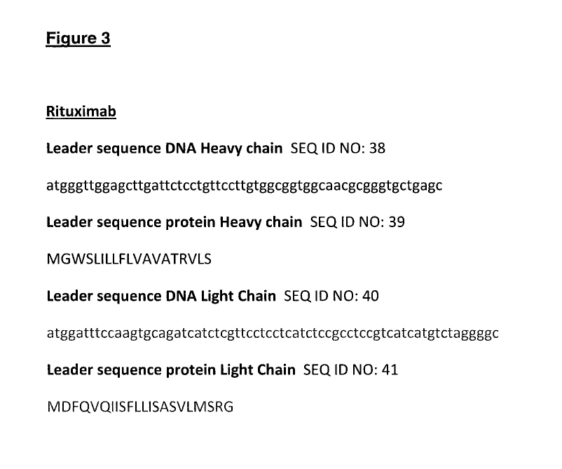

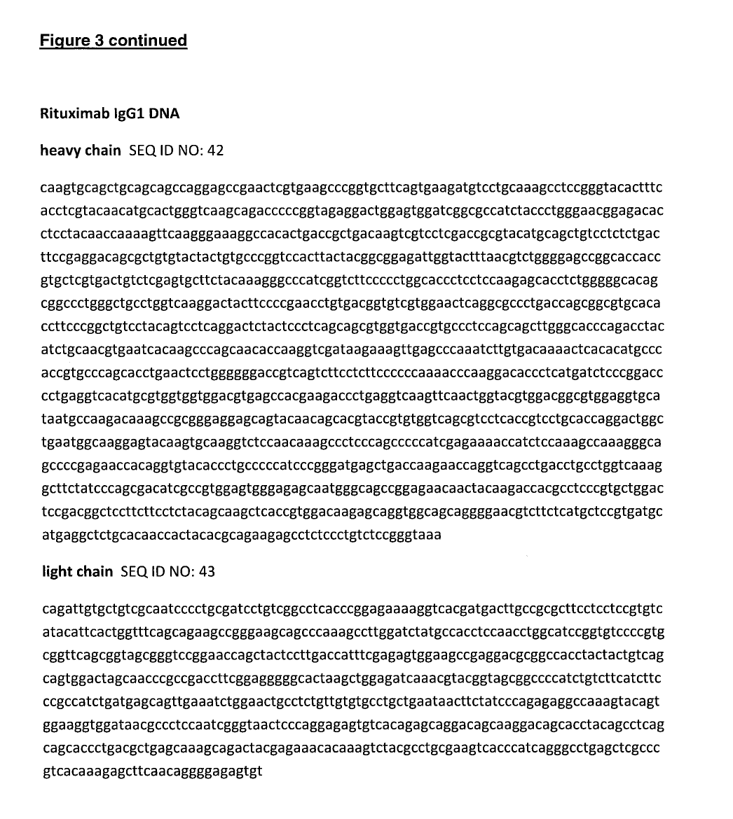

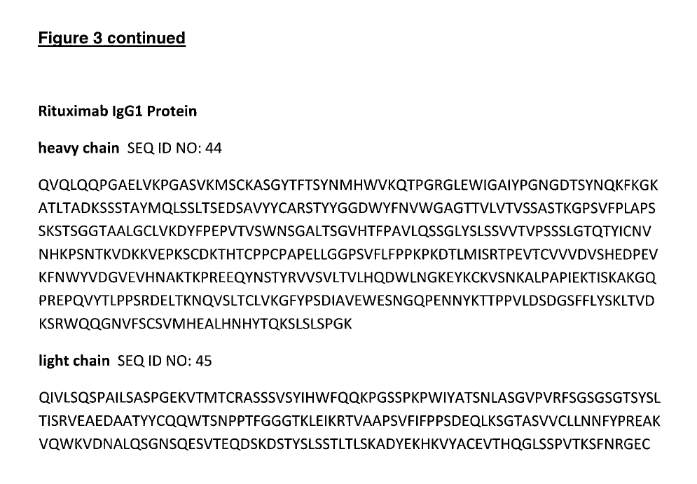

Diagrams showing example amino acid sequences of a polypeptide chain of a monomeric fusion protein are provided in FIG. 2. In each sequence, the tailpiece sequence is underlined, and any mutations are shown in bold and underlined. The hinge is in bold. In constructs comprising a CH4 domain from IgM, this region is shown in italics.

### Example 2

### Expression

Small scale expression was performed using ‘transient’ expression of HEK293 or CHO cells transfected using lipofectamine or electroporation. Cultures were grown in shaking flasks or agitated bags in CD-CHO (Lonza) or ProCHO5 (Life Technologies) media at scales ranging from 50-2000 ml for 5-10 days. Cells were removed by centrifugation and culture supernatants were stored at 4° C. until purified. Preservatives were added to some cultures after removal of cells.

### Example 3

### Purification and Analysis

Proteins were purified from culture supernatants after checking/adjusting pH to be ≥6.5, by protein A chromatography with step elution using a pH3.4 buffer. Eluate was immediately neutralised to ˜pH7.0 using 1M Tris pH8.5.

Samples were concentrated in 0.1M sodium citrate buffer pH7.5 using a pressure stirred cell, and 10 kDa cut-off membrane to >100 mg/ml. Samples were then diluted in PBS to a range of different final concentrations as shown in Table 3, before SE-HPLC analysis of multimeric form as described below. Endotoxin was tested using the limulus amoebocyte lysate (LAL) assay. Samples used in assays were <1 EU/mg.

SE-HPLC Analysis of Multimeric Form

TSK-G3000

Proteins were analysed using size exclusion HPLC, the column used was 15 ml TSKgel -G3000SW (Tosoh) on system Agilent 1100 Series. The mobile phase was 0.2 M sodium phosphate, pH7.0, flow rate 1 ml/minute, 50 μg protein injected. Signal was detected using a UV absorbance detector at 280 nm wavelength.

uPLC

Proteins were analysed using size exclusion HPLC, the column used was 2.5 ml BEH 200 (Waters) on system Agilent 1100 Series. The mobile phase was 0.2 M sodium phosphate, pH7.0, flow rate 0.4 ml/minute, 2.5 and 5 μg protein injected. Signal was detected using a Fluorescence detector Excitation: 350 nm, Emission: 390 nm.

Superdex 200 SEC-MALS

Proteins were analysed using size exclusion HPLC, the column used was 24 ml Superdex 200 10/300 (GE) on system Agilent 1100 Series. The mobile phase was 10 mM HEPES, 100 mM NaCI pH7.5, flow rate 0.5 ml/minute, 100 μg protein injected. Signal was detected using a UV absorbance detector at 280 nm wavelength and additional refractive index detector (Viscotek VE3580) and MALS detector (Viscotech SEC-MALS 20, Malvern)

Results

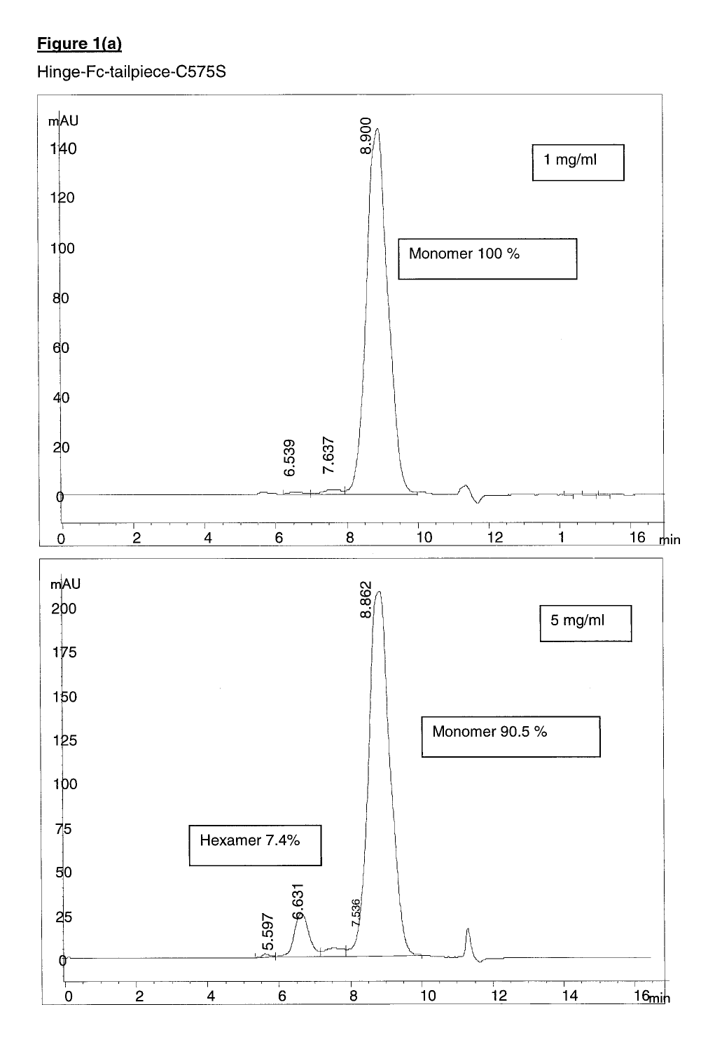

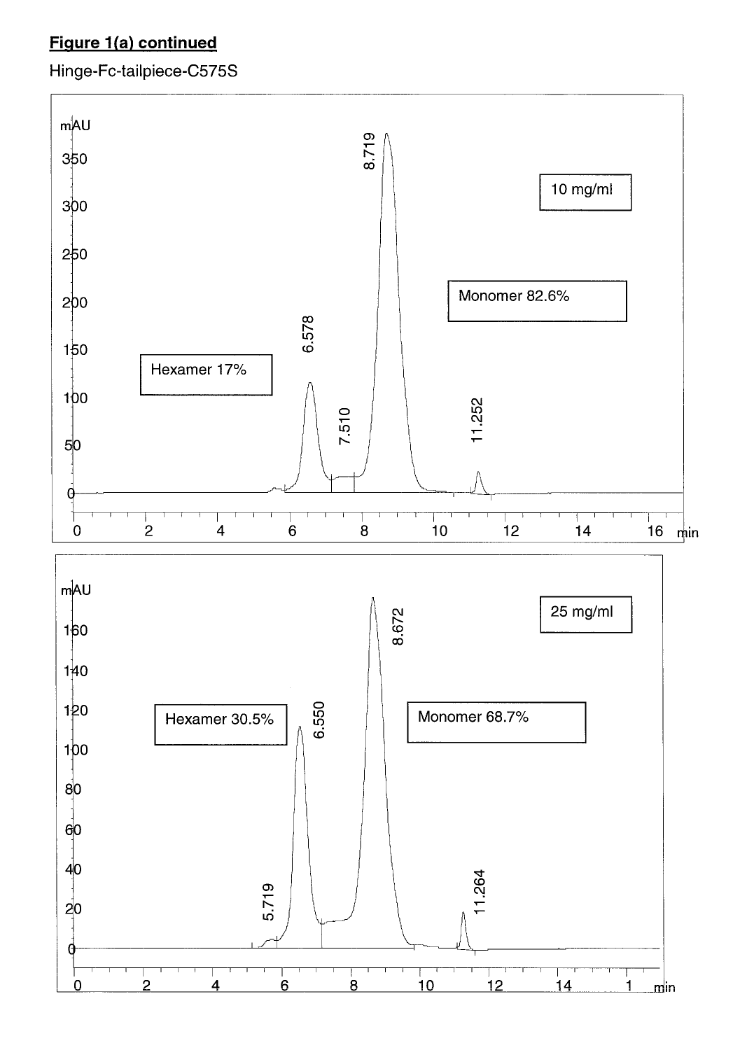

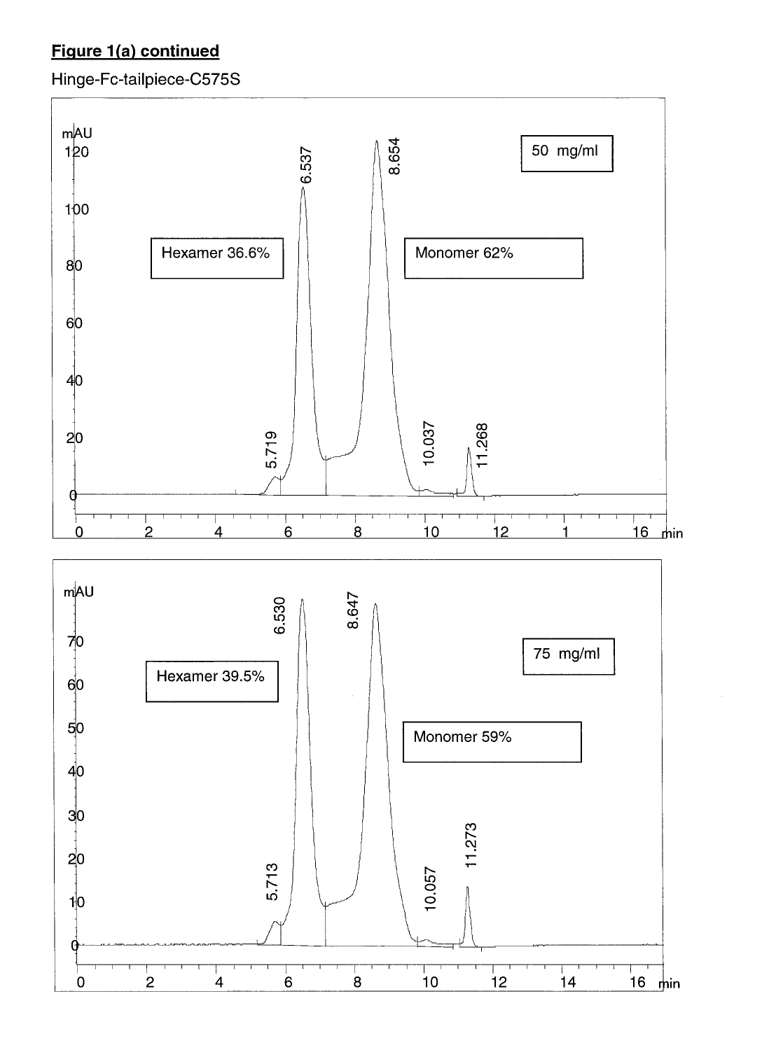

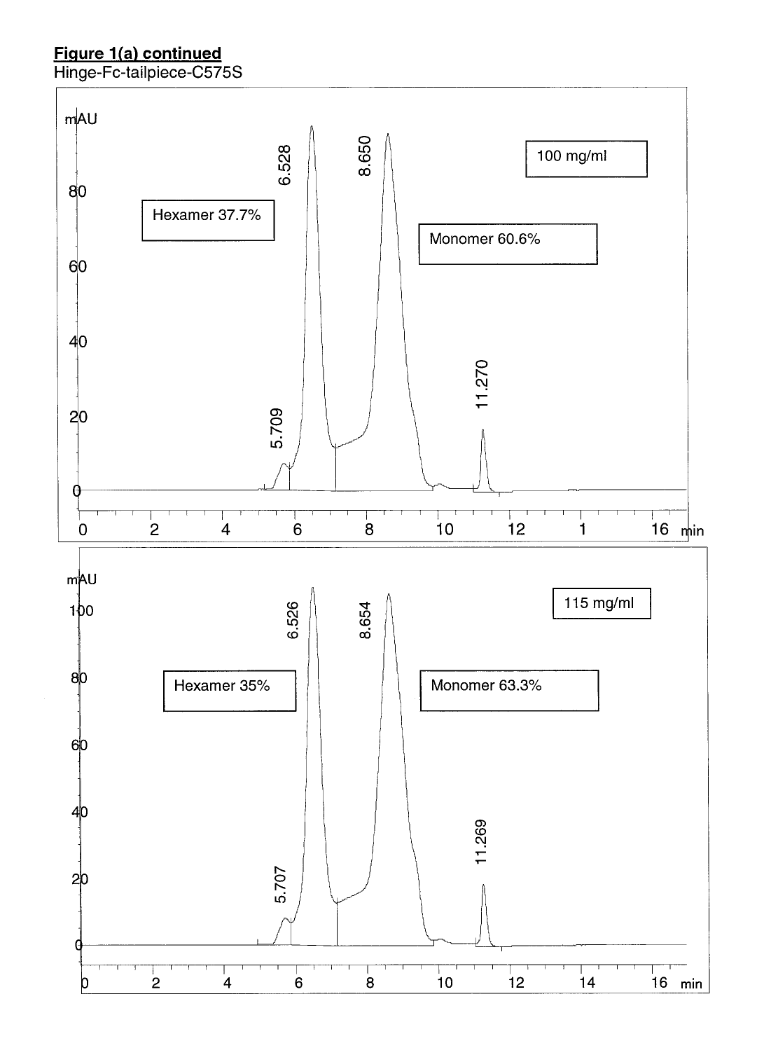

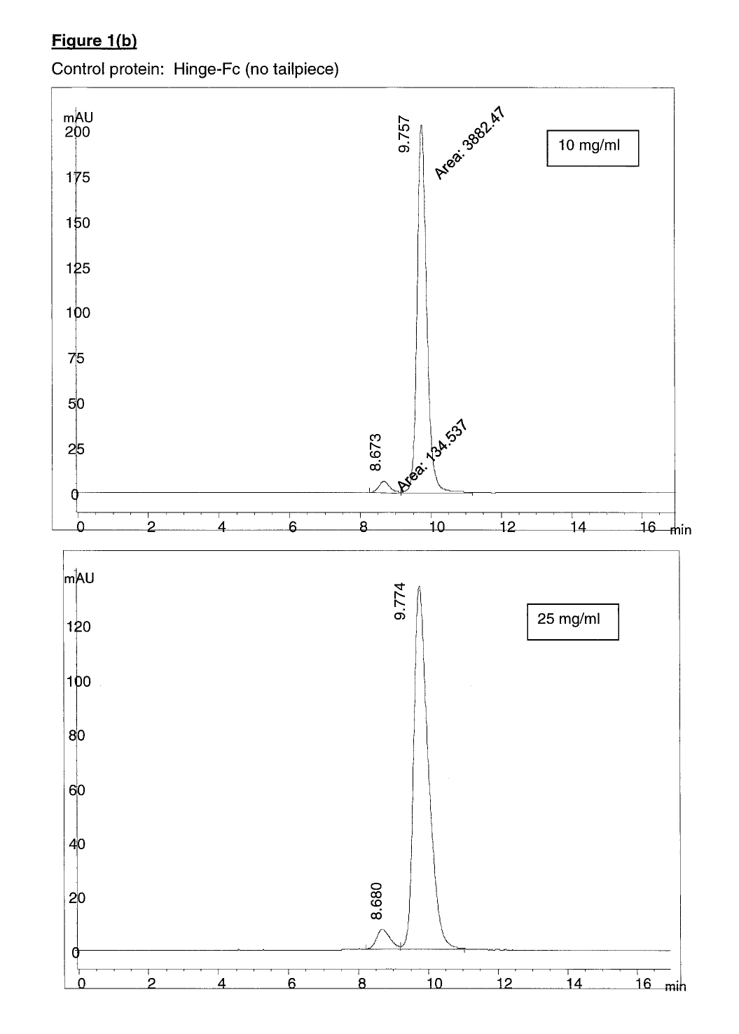

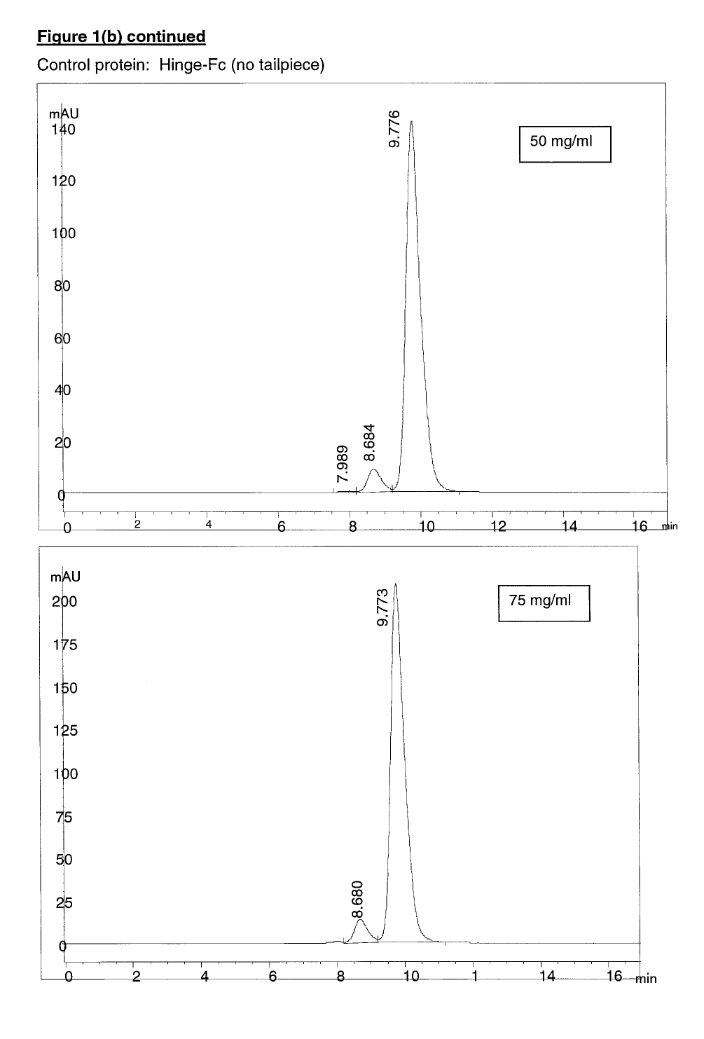

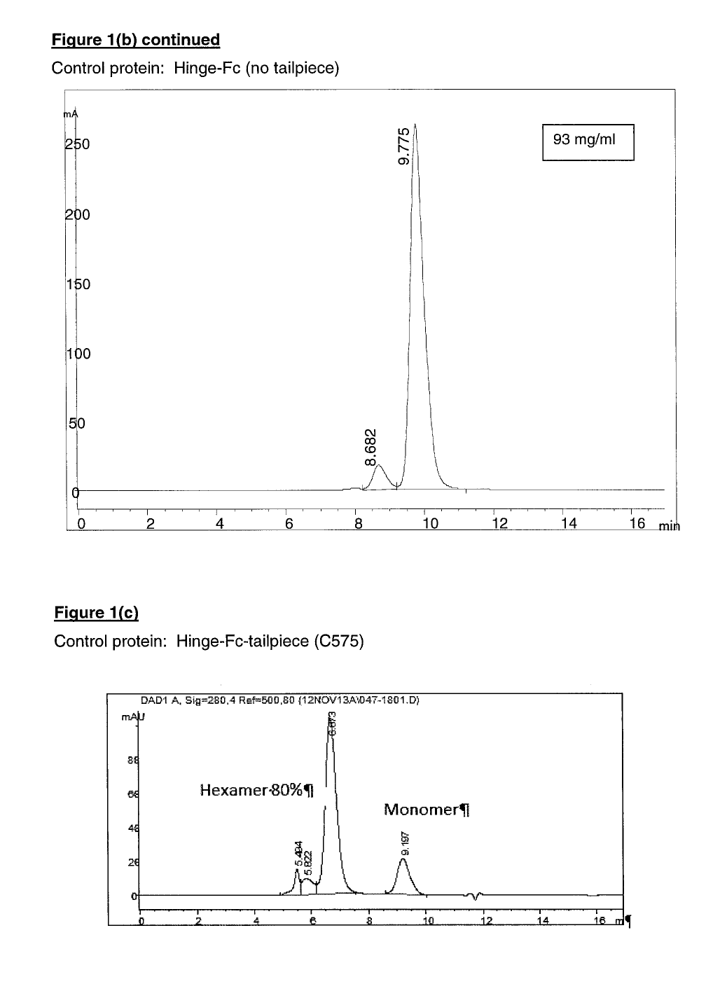

The results demonstrated that the fusion proteins of the invention are synthesised predominantly as monomers, and are capable of assembly into multimers at high concentrations, as shown in FIG. 1.

Hinge-Fc-tailpiece-05755:

Fusion proteins in which the tailpiece cysteine residue has been deleted or substituted with another amino acid residue are expressed predominantly as monomers and are capable of concentration-dependent assembly into hexamers. FIG. 1(a).

Control protein: Hinge-Fc (no tailpiece):

Control proteins lacking the modified tailpiece are unable to assemble into hexamers, even at the highest concentrations tested. FIG. 1(b).

Control protein: Hinge-Fc-tailpiece (C575):

Control proteins comprising an unmodified tailpiece with an intact cysteine residue are expressed predominantly in hexameric form. FIG. 1(c).

### Example 5

### Complement Dependent Cytotoxity (CDC)

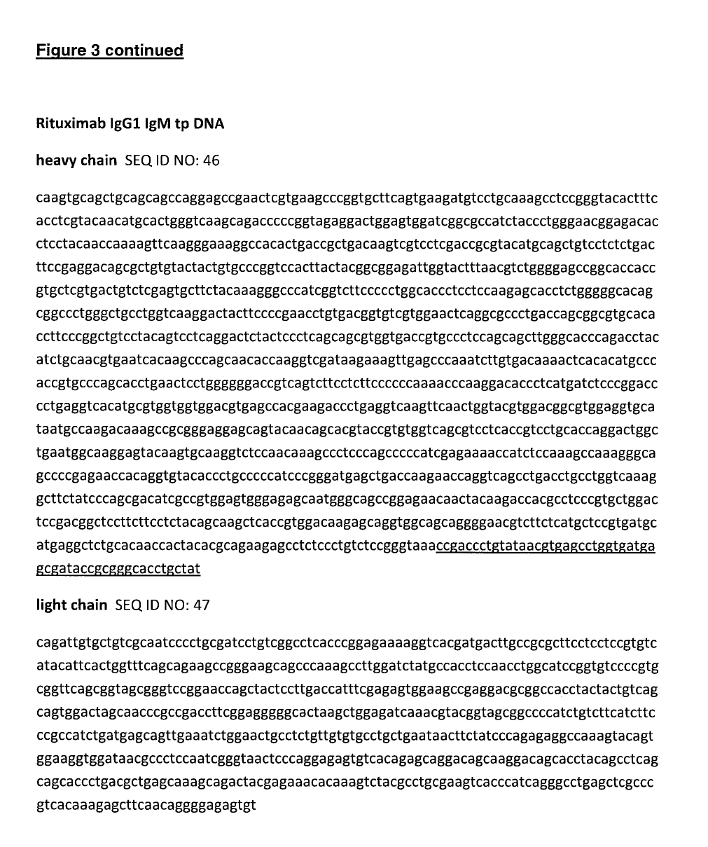

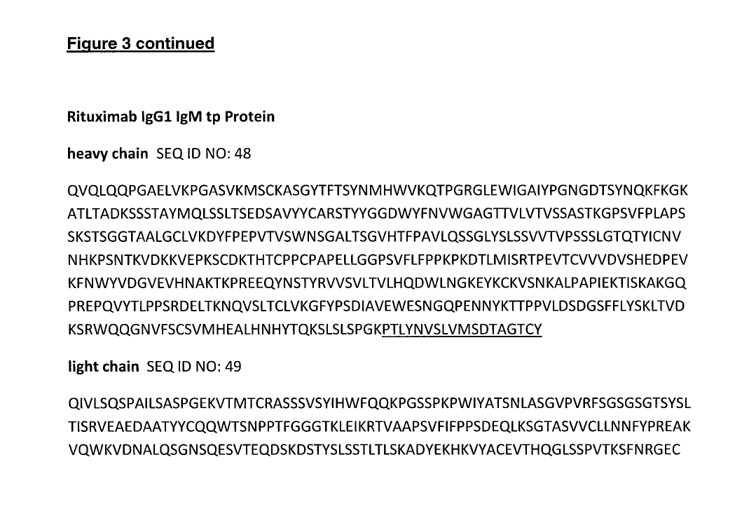

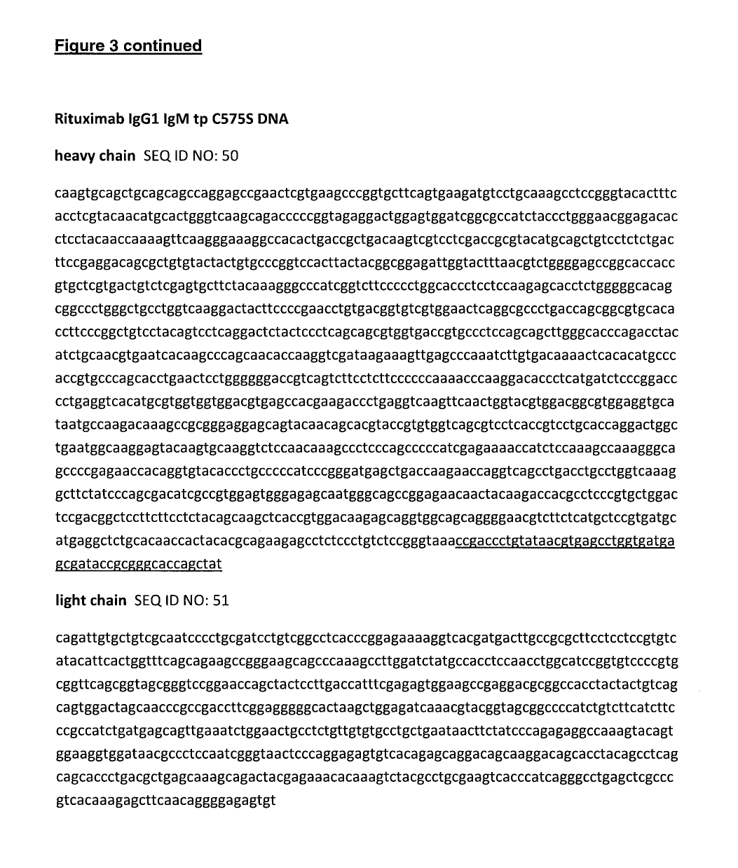

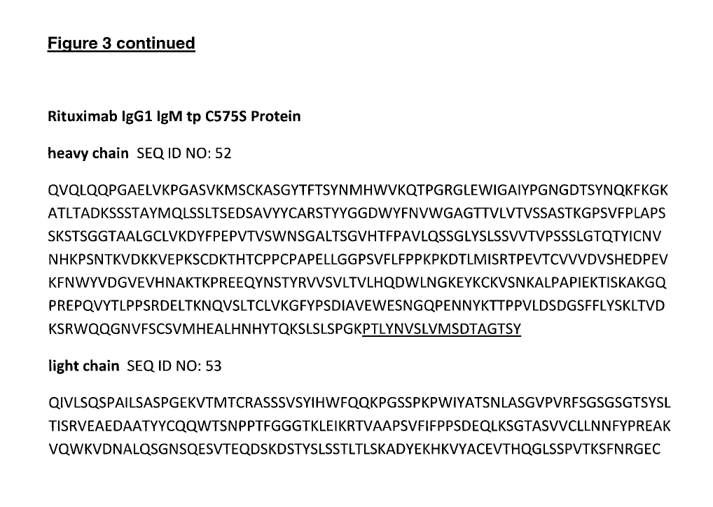

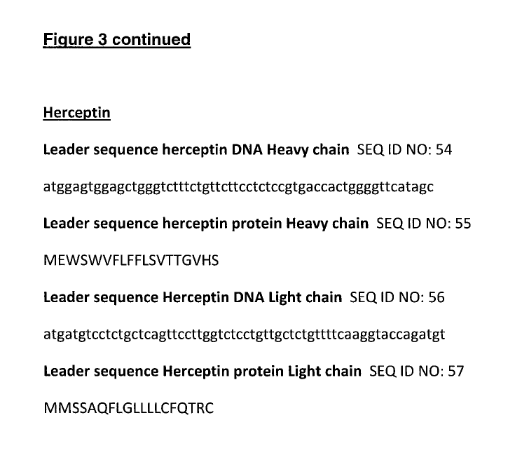

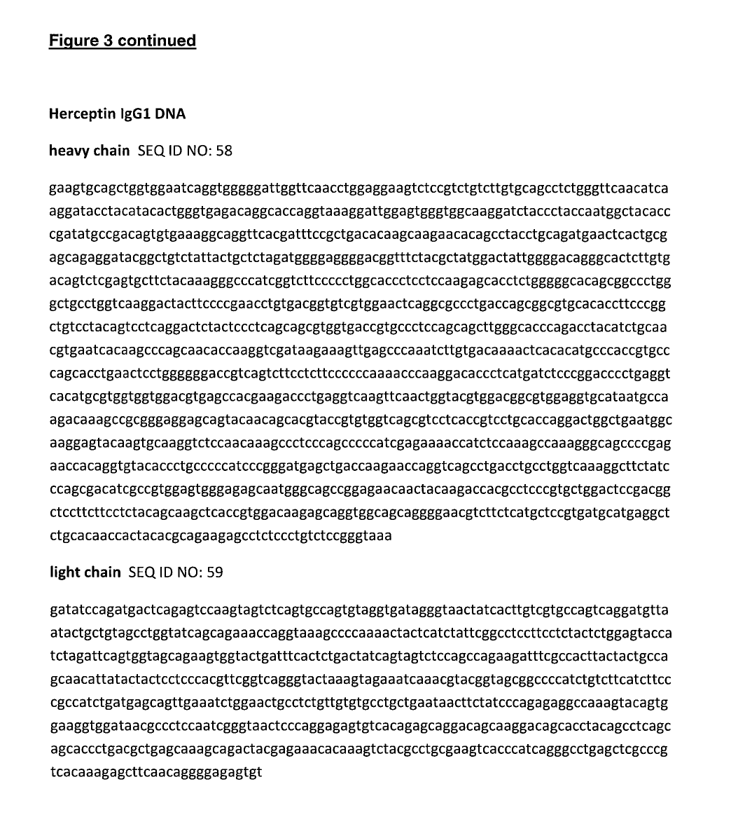

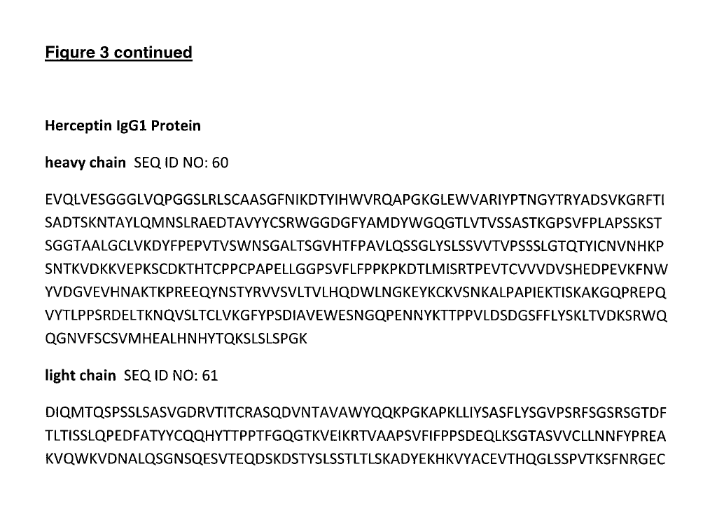

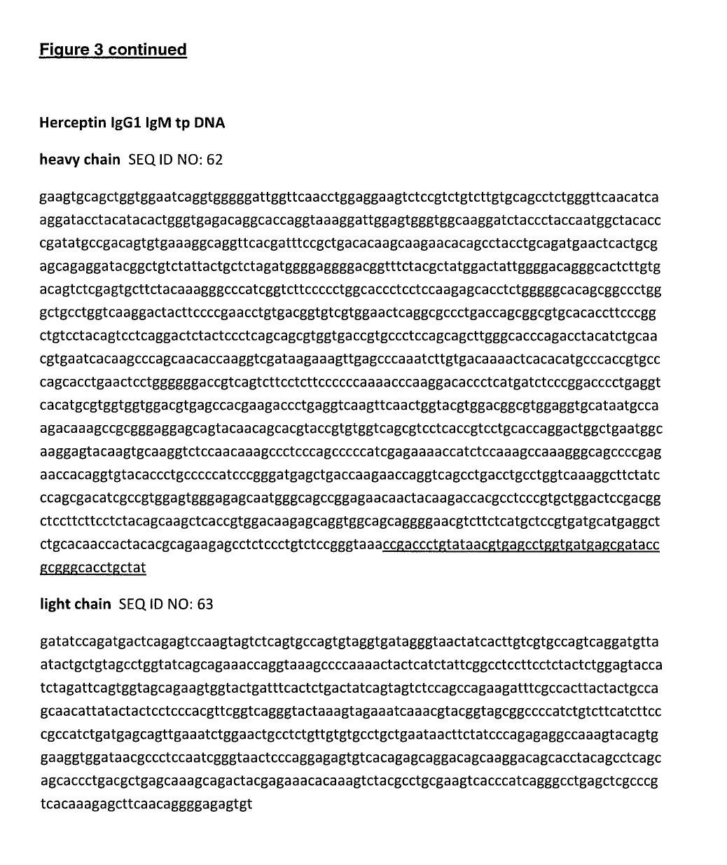

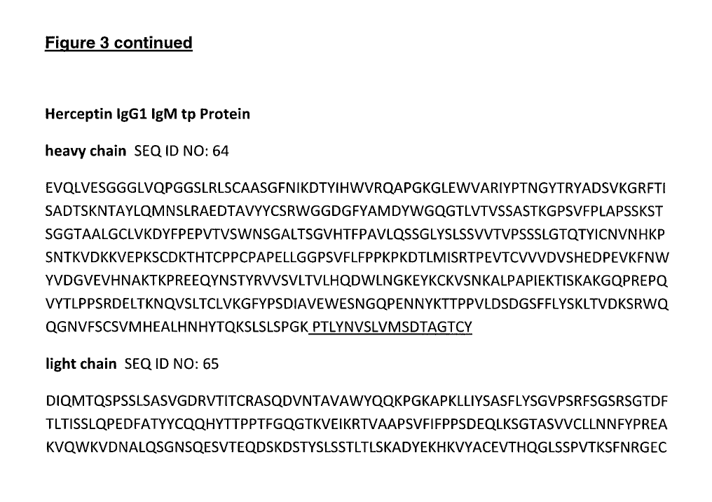

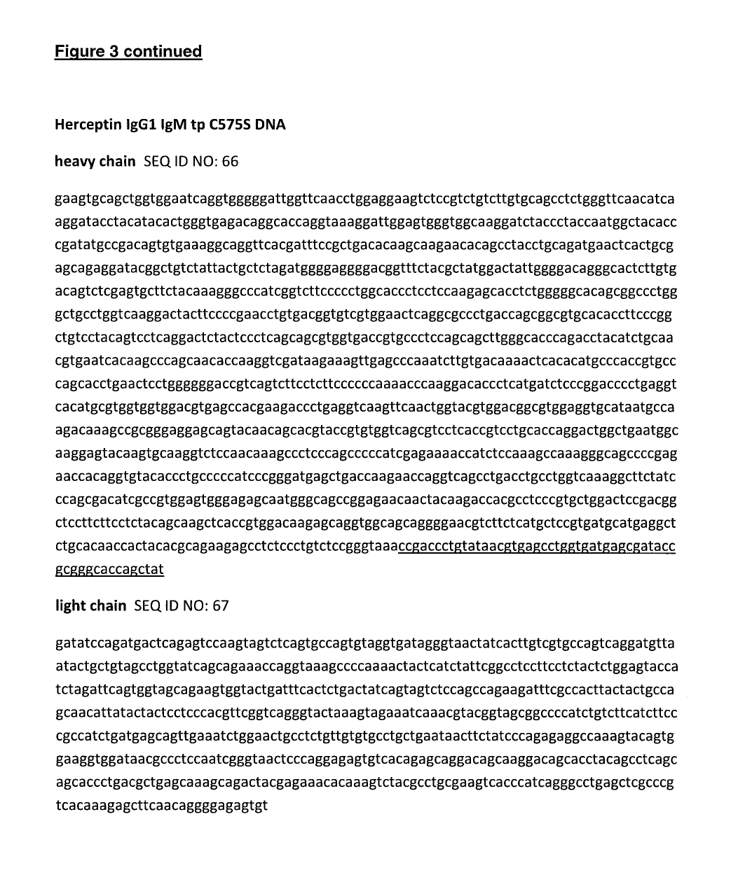

Full-length antibody constructs were prepared as shown in Table 3 below. Rituximab and trastuzumab are well-known antibodies that recognise CD20 and HER2/neu respectively. CA1151 recognises a clostridium difficile exotoxin and was included in the present study for comparison as a negative control antibody. Amino acid and DNA sequences for the antibody constructs are provided in FIG. 3.

TABLE 3
Antibody construct

rituximab IgG1 IgM tailpiece C575
rituximab IgG1 IgM tailpiece C575S
rituximab IgG1 wild type
CA1151 IgG1 IgM tailpiece C575
CA1151 IgG1 IgM tailpiece C575S
CA1151 IgG1 wild type
trastuzumab IgG1 IgM tailpiece C575
trastuzumab IgG1 IgM tailpiece C575S
trastuzumab IgG1 wild type

Concentration and Analytical HPLC

Proteins were concentrated by centrifugation using Amicon Ultra-15 Centrifugal Filter Units. Samples were centrifuged at 4000 RPM until desired concentration was reached. Proteins were analysed using size exclusion HPLC, the column used was 15 ml TSKgel -G3000SW (Tosoh) on system Agilent 1100 Series. The mobile phase was 0.2 M sodium phosphate, pH7.0, flow rate 1 ml/minute, 50 μg protein injected. Signal was detected using a UV absorbance detector at 280 nm wavelength.

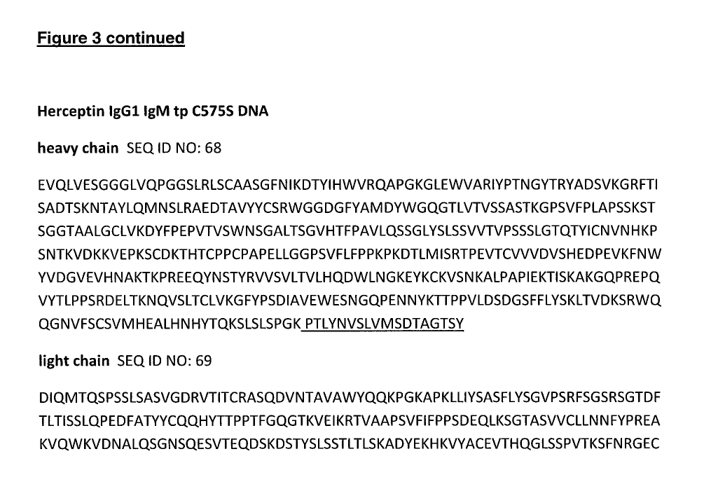

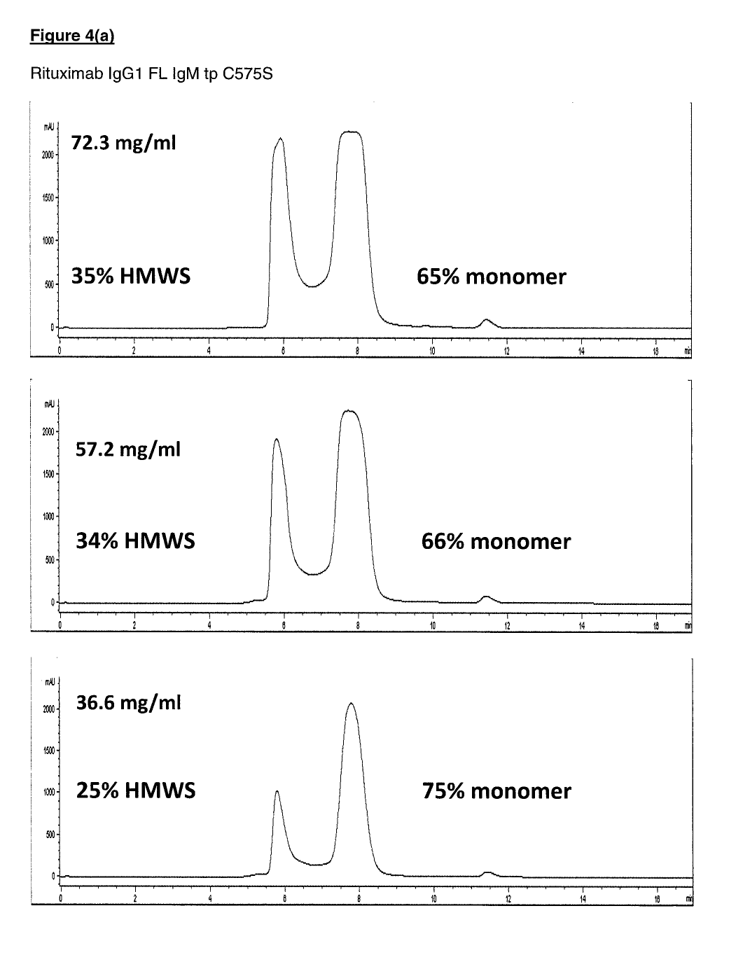

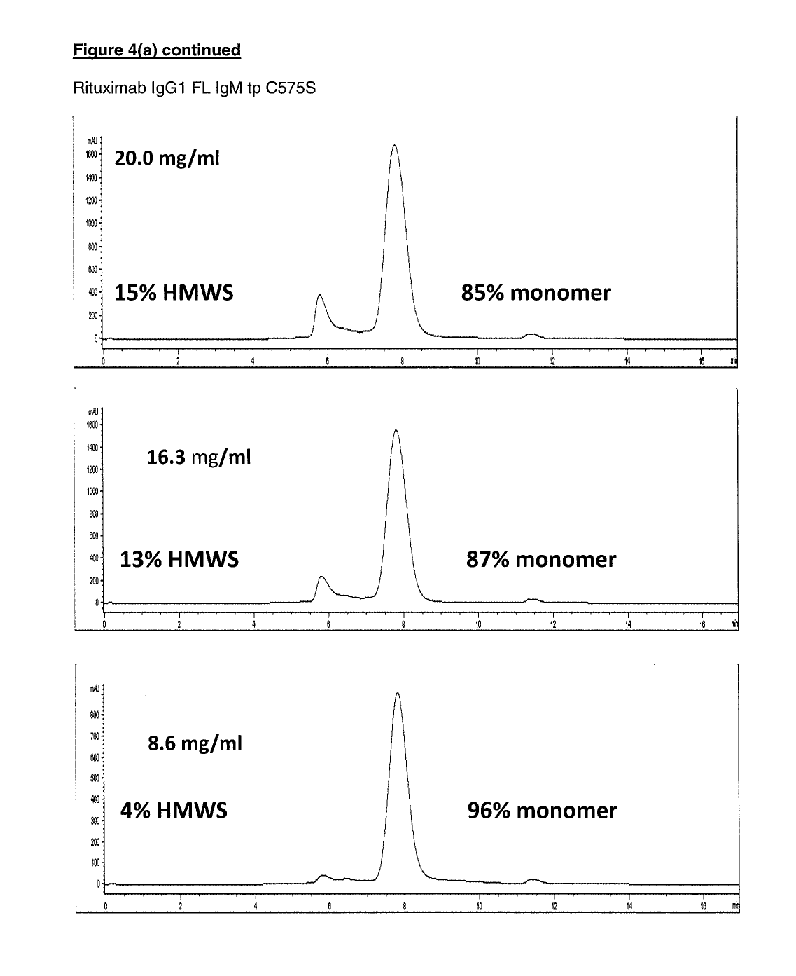

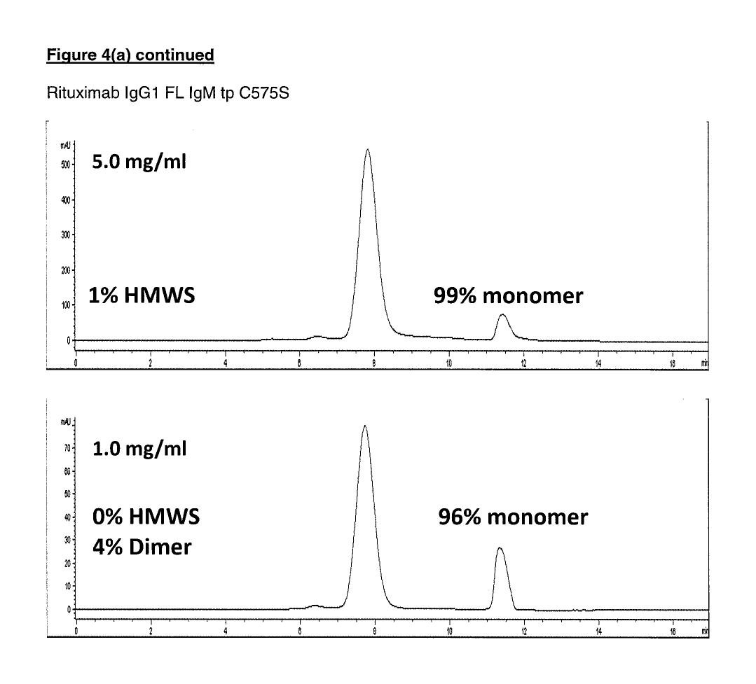

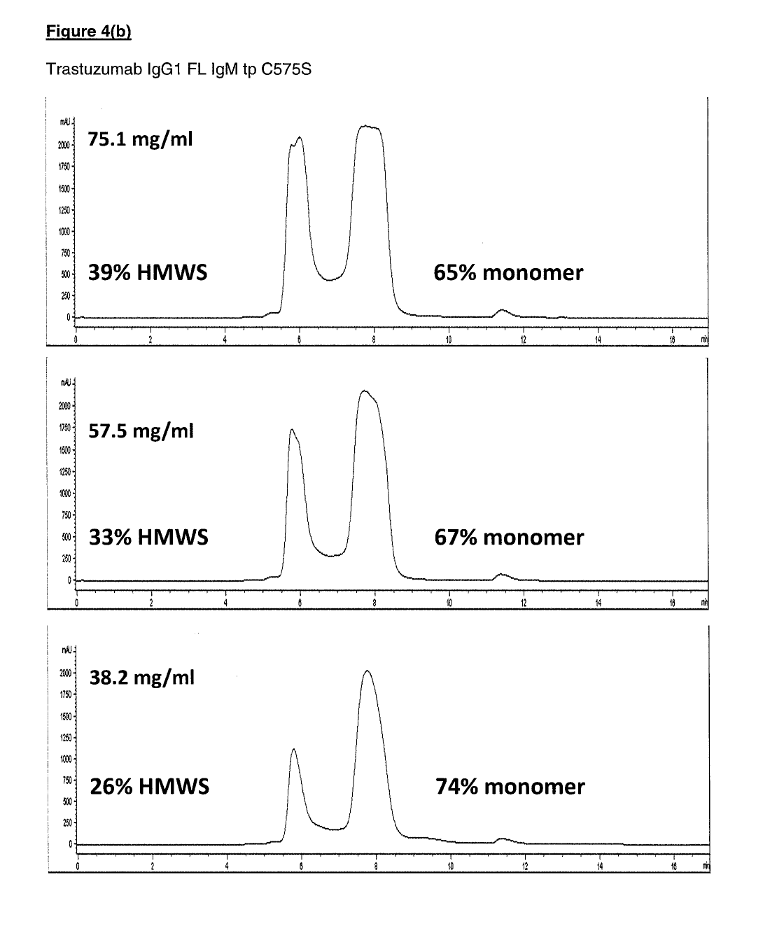

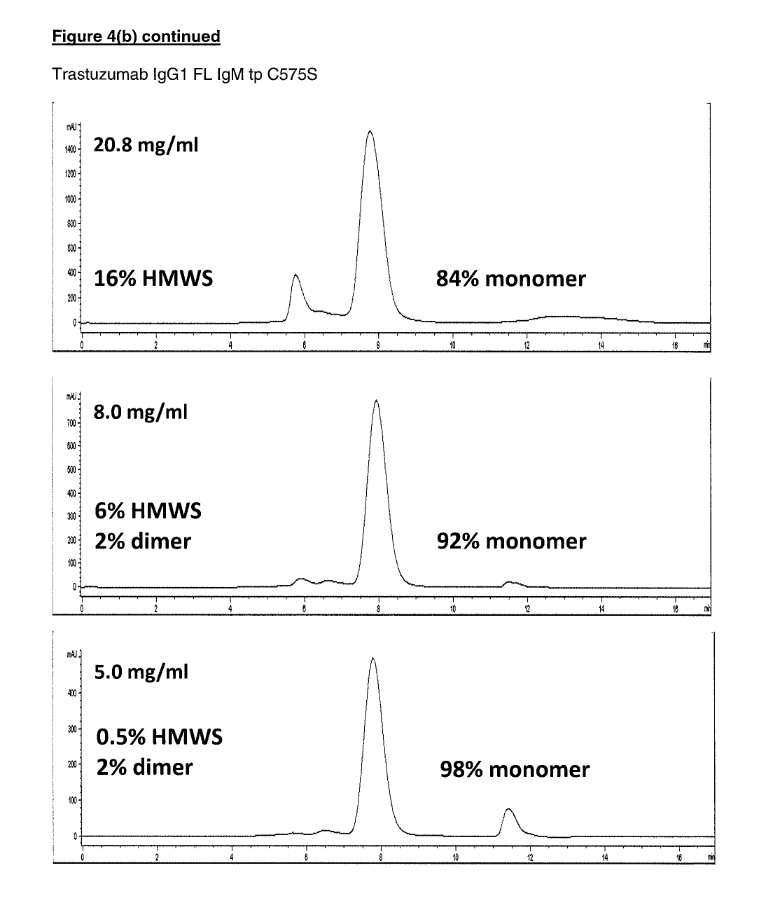

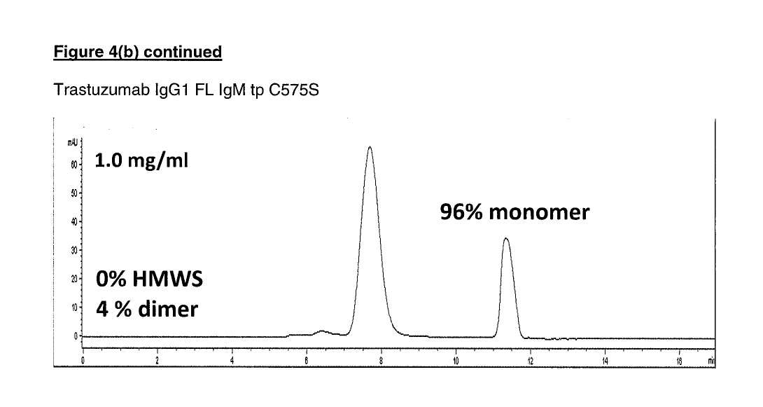

The results demonstrated that full length antibody fusion proteins comprising the modified tailpiece of the invention exhibit concentration-dependent multimerisation into a high molecular weight species (HMWS). FIG. 4.

CDC Assay

The biological activity of the modified antibodies was examined using a complement dependent cytotoxicity assay. Target cells (50×10⁵) were incubated with an antibody concentration series in a flat-bottom 96-well plate at a final volume of 100 μl, and allowed to opsonise for 15 minutes at room temperature. Opsonised target cells were incubated with normal human serum at a final concentration of 30% (42 μl added) and incubated in a 37° C. incubator for 30 minutes. Cell lysis measured as a fraction of P1+ cells determined by BD FACSCalibur flow cytometer. Graphs were plotted using GraphPad Prism software.

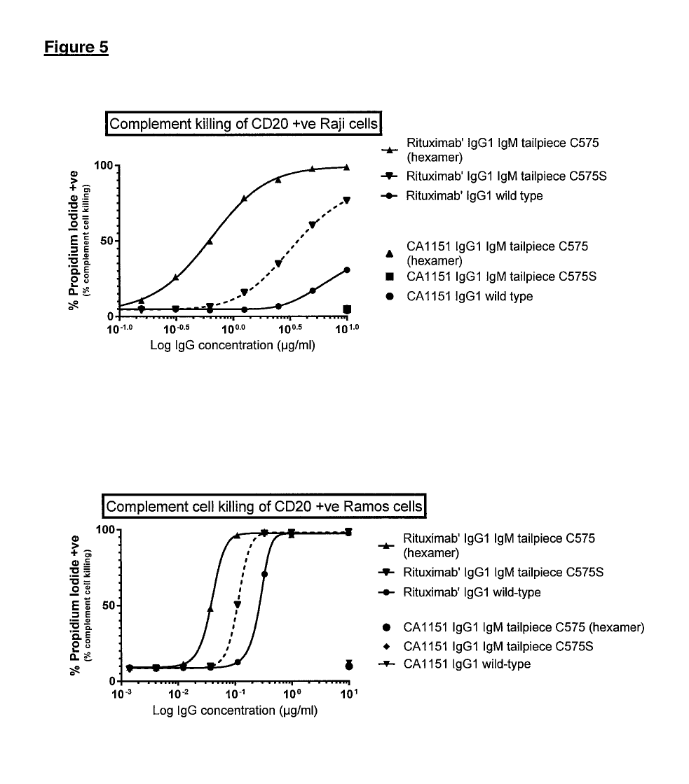

Results for a fusion protein comprising the anti-CD20 antibody rituximab are shown in FIG. 5. The graphs show complement killing of CD20 positive Raji cells and CD20 positive Ramos cells. The results demonstrated that rituximab shows enhanced CDC when modified with the tailpiece of the invention.

1. A monomeric fusion protein comprising an antibody Fc-domain comprising two heavy chain Fc-regions derived from IgG;
wherein one or each heavy chain Fc-region is fused at its C-terminal to an antibody tailpiece; and
wherein the tailpiece cysteine residue is modified to prevent disulphide bond formation.

2. The monomeric fusion protein of claim 1, wherein the cysteine residue is deleted, substituted, or blocked with a thiol capping agent.

3. The monomeric fusion protein of claim 1, further comprising an antigen binding region.

4. The monomeric fusion protein of claim 3, wherein the antigen binding region is a VH or a VL antigen binding region.

5. The monomeric fusion protein of claim 3, wherein the antigen binding region is selected from the group consisting of Fab, scFv, dAb, VHH, and DARPin.

6. The monomeric fusion protein of claim 1, further comprising a fusion partner.

7. The monomeric fusion protein of claim 6, wherein the fusion partner is selected from the group consisting of antigen, pathogen-associated molecular pattern (PAMP), drug, ligand, receptor, cytokine or chemokine.

8. The monomeric fusion protein of claim 1, wherein the heavy chain Fc-region comprises CH2 and CH3 domains derived from IgG1, IgG2, IgG3, or IgG4.

9. The monomeric fusion protein of claim 1, wherein the tailpiece is derived from human IgM or IgA.

10. The monomeric fusion protein of claim 1, wherein each heavy chain Fc-region possesses a hinge region at its N-terminus.

11. The monomeric fusion protein of claim 10, wherein the hinge region comprises the mutated sequence CPPC.

12. The monomeric fusion protein of claim 1, comprising one or more mutations which alter its Fc-receptor binding profile.

13. The monomeric fusion protein of claim 1 wherein each heavy chain Fc-region comprises or consists of the sequence given in amino acids 6 to 222 of any one of SEQ ID NOs 26 to 29, or the sequence given in amino acids 6 to 333 of SEQ ID NOs 30 or 31, or the sequence given in amino acids 6 to 221 of any one of SEQ ID NOs 32 to 35, or the sequence given in amino acids 6 to 332 of SEQ ID NOs 36 or 37.

14. The monomeric fusion protein of claim 13 wherein each heavy chain Fc-region further comprises a hinge region having a sequence given in any one of SEQ ID NOs: 3 to 25.

15. The monomeric fusion protein of claim 1 wherein each polypeptide monomer unit comprises or consists of two identical polypeptide chains each polypeptide chain comprising or consisting of the sequence given in any one of SEQ ID NOs 26 to 37.

16. The monomeric fusion protein of claim 1 which is a purified monomer.

17. A mixture comprising a monomeric fusion protein according to claim 1 and a multimer, said multimer comprising two or more monomer units.

18. The mixture of claim 17, comprising greater than 55% monomer.

19. An isolated DNA sequence encoding a polypeptide chain of a monomeric fusion protein according to claim 1, or a component part thereof.

20. A cloning or expression vector comprising one or more DNA sequences according to claim 19.

21. A host cell comprising one or more cloning or expression vectors according to claim 20.

22. A process for the production of a monomeric fusion protein according to claim 1, comprising culturing a host cell comprising one or more cloning or expression vectors comprising one or more DNA sequences encoding a polypeptide chain of a monomeric fusion protein, or a component part thereof, wherein the monomeric fusion protein comprises an antibody Fc-domain comprising two heavy chain Fc-regions derived from IgG;
wherein one or each heavy chain Fc-region is fused at its C-terminal to an antibody tailpiece; and wherein the tailpiece cysteine residue is modified to prevent disulphide bond formation under conditions suitable for protein expression, and isolating and optionally purifying the monomeric fusion protein.

23. A pharmaceutical composition comprising a monomeric fusion protein of claim 1, in combination with a pharmaceutically acceptable excipient, diluent or carrier.

24. The pharmaceutical composition of claim 23, comprising a component that stabilises the monomeric form of the protein or increases the ratio of monomer to multimer in a mixture.

25-27. (canceled)

28. A method of treating cancer comprising administering the monomeric fusion protein of claim 1 to a subject in need thereof.

29. (canceled)

30. A method of treating immune disorders comprising administering the monomeric fusion protein of claim 1 to a subject in need thereof.

31. (canceled)

32. A method of treating cancer or immune disorders comprising administering the pharmaceutical composition of claim 23 to a subject in need thereof.


SEQUENCE LISTING (69 sequences)

SEQ ID NO: 1 - protein, 18 aa, Artificial Sequence
  recombinant sequence
PTLYNVSLVMSDTAGTCY

SEQ ID NO: 2 - protein, 18 aa, Artificial Sequence
  recombinant sequence
PTHVNVSVVMAEVDGTCY

SEQ ID NO: 3 - protein, 19 aa, Artificial Sequence
  recombinant sequence
VPSTPPTPSPSTPPTPSPS

SEQ ID NO: 4 - protein, 6 aa, Artificial Sequence
  recombinant sequence
VPPPPP

SEQ ID NO: 5 - protein, 58 aa, Artificial Sequence
  recombinant sequence
ESPKAQASSVPTAQPQAEGSLAKATTAPATTRNTGRGGEEKKKEKEKEEQEERETKTP

SEQ ID NO: 6 - protein, 15 aa, Artificial Sequence
  recombinant sequence
EPKSCDKTHTCPPCP

SEQ ID NO: 7 - protein, 12 aa, Artificial Sequence
  recombinant sequence
ERKCCVECPPCP

SEQ ID NO: 8 - protein, 62 aa, Artificial Sequence
  recombinant sequence
ELKTPLGDTTHTCPRCPEPKSCDTPPPCPRCPEPKSCDTPPPCPRCPEPKSCDTPPPCPRCP

SEQ ID NO: 9 - protein, 12 aa, Artificial Sequence
  recombinant sequence
ESKYGPPCPSCP

SEQ ID NO: 10 - protein, 12 aa, Artificial Sequence
  recombinant sequence
ESKYGPPCPPCP

SEQ ID NO: 11 - protein, 4 aa, Artificial Sequence
  recombinant sequence
CPPC

SEQ ID NO: 12 - protein, 4 aa, Artificial Sequence
  recombinant sequence
CPSC

SEQ ID NO: 13 - protein, 4 aa, Artificial Sequence
  recombinant sequence
CPRC

SEQ ID NO: 14 - protein, 4 aa, Artificial Sequence
  recombinant sequence
SPPC

SEQ ID NO: 15 - protein, 4 aa, Artificial Sequence
  recombinant sequence
CPPS

SEQ ID NO: 16 - protein, 4 aa, Artificial Sequence
  recombinant sequence
SPPS

SEQ ID NO: 17 - protein, 8 aa, Artificial Sequence
  recombinant sequence
DKTHTCAA

SEQ ID NO: 18 - protein, 11 aa, Artificial Sequence
  recombinant sequence
DKTHTCPPCPA

SEQ ID NO: 19 - protein, 18 aa, Artificial Sequence
  recombinant sequence
DKTHTCPPCPATCPPCPA

SEQ ID NO: 20 - protein, 25 aa, Artificial Sequence
  recombinant sequence
DKTHTCPPCPATCPPCPATCPPCPA

SEQ ID NO: 21 - protein, 30 aa, Artificial Sequence
  recombinant sequence
DKTHTCPPCPAGKPTLYNSLVMSDTAGTCY

SEQ ID NO: 22 - protein, 31 aa, Artificial Sequence
  recombinant sequence
DKTHTCPPCPAGKPTHVNVSVVMAEVDGTCY

SEQ ID NO: 23 - protein, 15 aa, Artificial Sequence
  recombinant sequence
DKTHTCCVECPPCPA

SEQ ID NO: 24 - protein, 26 aa, Artificial Sequence
  recombinant sequence
DKTHTCPRCPEPKSCDTPPPCPRCPA

SEQ ID NO: 25 - protein, 11 aa, Artificial Sequence
  recombinant sequence
DKTHTCPSCPA

SEQ ID NO: 26 - protein, 240 aa, Artificial Sequence
  recombinant sequence
  misc_feature (239)..(239): Xaa can be any naturally occurring amino acid
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTXY

SEQ ID NO: 27 - protein, 240 aa, Artificial Sequence
  recombinant sequence
  misc_feature (239)..(239): Xaa can be any naturally occurring amino acid
CPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPTLYNVSLVMSDTAGTXY

SEQ ID NO: 28 - protein, 240 aa, Artificial Sequence
  recombinant sequence
  misc_feature (239)..(239): Xaa can be any naturally occurring amino acid
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTHVNVSVVMAEVDGTXY

SEQ ID NO: 29 - protein, 240 aa, Artificial Sequence
  recombinant sequence
  misc_feature (239)..(239): Xaa can be any naturally occurring amino acid
CPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPTHVNVSVVMAEVDGTXY

SEQ ID NO: 30 - protein, 351 aa, Artificial Sequence
  recombinant sequence
  misc_feature (350)..(350): Xaa can be any naturally occurring amino acid
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTXY

SEQ ID NO: 31 - protein, 351 aa, Artificial Sequence
  recombinant sequence
  misc_feature (350)..(350): Xaa can be any naturally occurring amino acid
CPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTXY

SEQ ID NO: 32 - protein, 239 aa, Artificial Sequence
  recombinant sequence
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTY

SEQ ID NO: 33 - protein, 239 aa, Artificial Sequence
  recombinant sequence
CPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPTLYNVSLVMSDTAGTY

SEQ ID NO: 34 - protein, 239 aa, Artificial Sequence
  recombinant sequence
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTHVNVSVVMAEVDGTY

SEQ ID NO: 35 - protein, 239 aa, Artificial Sequence
  recombinant sequence
CPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPTHVNVSVVMAEVDGTY

SEQ ID NO: 36 - protein, 350 aa, Artificial Sequence
  recombinant sequence
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTY

SEQ ID NO: 37 - protein, 350 aa, Artificial Sequence
  recombinant sequence
CPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTY

SEQ ID NO: 38 - DNA, 57 bp, Artificial Sequence
  recombinant sequence
atgggttggagcttgattctcctgttccttgtggcggtggcaacgcgggtgctgagc

SEQ ID NO: 39 - protein, 19 aa, Artificial Sequence
  recombinant sequence
MGWSLILLFLVAVATRVLS

SEQ ID NO: 40 - DNA, 66 bp, Artificial Sequence
  recombinant sequence
atggatttccaagtgcagatcatctcgttcctcctcatctccgcctccgtcatcatgtctaggggc

SEQ ID NO: 41 - protein, 22 aa, Artificial Sequence
  recombinant sequence
MDFQVQIISFLLISASVLMSRG

SEQ ID NO: 42 - DNA, 1359 bp, Artificial Sequence
  recombinant sequence
caagtgcagctgcagcagccaggagccgaactcgtgaagcccggtgcttcagtgaagatgtcctgcaaagcctccgggtacactttcacctcgtacaacatgcactgggtcaagcagacccccggtagaggactggagtggatcggcgccatctaccctgggaacggagacacctcctacaaccaaaagttcaagggaaaggccacactgaccgctgacaagtcgtcctcgaccgcgtacatgcagctgtcctctctgacttccgaggacagcgctgtgtactactgtgcccggtccacttactacggcggagattggtactttaacgtctggggagccggcaccaccgtgctcgtgactgtctcgagtgcttctacaaagggcccatcggtcttccccctggcaccctcctccaagagcacctctgggggcacagcggccctgggctgcctggtcaaggactacttccccgaacctgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgataagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 43 - DNA, 639 bp, Artificial Sequence
  recombinant sequence
cagattgtgctgtcgcaatcccctgcgatcctgtcggcctcacccggagaaaaggtcacgatgacttgccgcgcttcctcctccgtgtcatacattcactggtttcagcagaagccgggaagcagcccaaagccttggatctatgccacctccaacctggcatccggtgtccccgtgcggttcagcggtagcgggtccggaaccagctactccttgaccatttcgagagtggaagccgaggacgcggccacctactactgtcagcagtggactagcaacccgccgaccttcggagggggcactaagctggagatcaaacgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 44 - protein, 453 aa, Artificial Sequence
  recombinant sequence
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 213 aa, Artificial Sequence
  recombinant sequence
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - DNA, 1413 bp, Artificial Sequence
  recombinant sequence
caagtgcagctgcagcagccaggagccgaactcgtgaagcccggtgcttcagtgaagatgtcctgcaaagcctccgggtacactttcacctcgtacaacatgcactgggtcaagcagacccccggtagaggactggagtggatcggcgccatctaccctgggaacggagacacctcctacaaccaaaagttcaagggaaaggccacactgaccgctgacaagtcgtcctcgaccgcgtacatgcagctgtcctctctgacttccgaggacagcgctgtgtactactgtgcccggtccacttactacggcggagattggtactttaacgtctggggagccggcaccaccgtgctcgtgactgtctcgagtgcttctacaaagggcccatcggtcttccccctggcaccctcctccaagagcacctctgggggcacagcggccctgggctgcctggtcaaggactacttccccgaacctgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgataagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaccgaccctgtataacgtgagcctggtgatgagcgataccgcgggcacctgctat

SEQ ID NO: 47 - DNA, 639 bp, Artificial Sequence; identical to SEQ ID NO: 43
  recombinant sequence

SEQ ID NO: 48 - protein, 471 aa, Artificial Sequence
  recombinant sequence
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 49 - protein, 213 aa, Artificial Sequence; identical to SEQ ID NO: 45
  recombinant sequence

SEQ ID NO: 50 - DNA, 1413 bp, Artificial Sequence
  recombinant sequence
caagtgcagctgcagcagccaggagccgaactcgtgaagcccggtgcttcagtgaagatgtcctgcaaagcctccgggtacactttcacctcgtacaacatgcactgggtcaagcagacccccggtagaggactggagtggatcggcgccatctaccctgggaacggagacacctcctacaaccaaaagttcaagggaaaggccacactgaccgctgacaagtcgtcctcgaccgcgtacatgcagctgtcctctctgacttccgaggacagcgctgtgtactactgtgcccggtccacttactacggcggagattggtactttaacgtctggggagccggcaccaccgtgctcgtgactgtctcgagtgcttctacaaagggcccatcggtcttccccctggcaccctcctccaagagcacctctgggggcacagcggccctgggctgcctggtcaaggactacttccccgaacctgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgataagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaccgaccctgtataacgtgagcctggtgatgagcgataccgcgggcaccagctat

SEQ ID NO: 51 - DNA, 639 bp, Artificial Sequence; identical to SEQ ID NO: 43
  recombinant sequence

SEQ ID NO: 52 - protein, 471 aa, Artificial Sequence
  recombinant sequence
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTSY

SEQ ID NO: 53 - protein, 213 aa, Artificial Sequence; identical to SEQ ID NO: 45
  recombinant sequence

SEQ ID NO: 54 - DNA, 57 bp, Artificial Sequence
  recombinant sequence
atggagtggagctgggtctttctgttcttcctctccgtgaccactggggttcatagc

SEQ ID NO: 55 - protein, 19 aa, Artificial Sequence
  recombinant sequence
MEWSWVFLFFLSVTTGVHS

SEQ ID NO: 56 - DNA, 60 bp, Artificial Sequence
  recombinant sequence
atgatgtcctctgctcagttccttggtctcctgttgctctgttttcaaggtaccagatgt

SEQ ID NO: 57 - protein, 19 aa, Artificial Sequence
  recombinant sequence
MMSSAQFLGLLLLCFQTRC

SEQ ID NO: 58 - DNA, 1350 bp, Artificial Sequence
  recombinant sequence
gaagtgcagctggtggaatcaggtgggggattggttcaacctggaggaagtctccgtctgtcttgtgcagcctctgggttcaacatcaaggatacctacatacactgggtgagacaggcaccaggtaaaggattggagtgggtggcaaggatctaccctaccaatggctacacccgatatgccgacagtgtgaaaggcaggttcacgatttccgctgacacaagcaagaacacagcctacctgcagatgaactcactgcgagcagaggatacggctgtctattactgctctagatggggaggggacggtttctacgctatggactattggggacagggcactcttgtgacagtctcgagtgcttctacaaagggcccatcggtcttccccctggcaccctcctccaagagcacctctgggggcacagcggccctgggctgcctggtcaaggactacttccccgaacctgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgataagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 59 - DNA, 642 bp, Artificial Sequence
  recombinant sequence
gatatccagatgactcagagtccaagtagtctcagtgccagtgtaggtgatagggtaactatcacttgtcgtgccagtcaggatgttaatactgctgtagcctggtatcagcagaaaccaggtaaagccccaaaactactcatctattcggcctccttcctctactctggagtaccatctagattcagtggtagcagaagtggtactgatttcactctgactatcagtagtctccagccagaagatttcgccacttactactgccagcaacattatactactcctcccacgttcggtcagggtactaaagtagaaatcaaacgtacggtagcggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 60 - protein, 450 aa, Artificial Sequence
  recombinant sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 214 aa, Artificial Sequence
  recombinant sequence
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - DNA, 1404 bp, Artificial Sequence
  recombinant sequence
gaagtgcagctggtggaatcaggtgggggattggttcaacctggaggaagtctccgtctgtcttgtgcagcctctgggttcaacatcaaggatacctacatacactgggtgagacaggcaccaggtaaaggattggagtgggtggcaaggatctaccctaccaatggctacacccgatatgccgacagtgtgaaaggcaggttcacgatttccgctgacacaagcaagaacacagcctacctgcagatgaactcactgcgagcagaggatacggctgtctattactgctctagatggggaggggacggtttctacgctatggactattggggacagggcactcttgtgacagtctcgagtgcttctacaaagggcccatcggtcttccccctggcaccctcctccaagagcacctctgggggcacagcggccctgggctgcctggtcaaggactacttccccgaacctgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgataagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaccgaccctgtataacgtgagcctggtgatgagcgataccgcgggcacctgctat

SEQ ID NO: 63 - DNA, 642 bp, Artificial Sequence; identical to SEQ ID NO: 59
  recombinant sequence

SEQ ID NO: 64 - protein, 468 aa, Artificial Sequence
  recombinant sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 65 - protein, 214 aa, Artificial Sequence; identical to SEQ ID NO: 61
  recombinant sequence

SEQ ID NO: 66 - DNA, 1404 bp, Artificial Sequence
  recombinant sequence
gaagtgcagctggtggaatcaggtgggggattggttcaacctggaggaagtctccgtctgtcttgtgcagcctctgggttcaacatcaaggatacctacatacactgggtgagacaggcaccaggtaaaggattggagtgggtggcaaggatctaccctaccaatggctacacccgatatgccgacagtgtgaaaggcaggttcacgatttccgctgacacaagcaagaacacagcctacctgcagatgaactcactgcgagcagaggatacggctgtctattactgctctagatggggaggggacggtttctacgctatggactattggggacagggcactcttgtgacagtctcgagtgcttctacaaagggcccatcggtcttccccctggcaccctcctccaagagcacctctgggggcacagcggccctgggctgcctggtcaaggactacttccccgaacctgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgataagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaccgaccctgtataacgtgagcctggtgatgagcgataccgcgggcaccagctat

SEQ ID NO: 67 - DNA, 642 bp, Artificial Sequence; identical to SEQ ID NO: 59
  recombinant sequence

SEQ ID NO: 68 - protein, 468 aa, Artificial Sequence
  recombinant sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTSY

SEQ ID NO: 69 - protein, 214 aa, Artificial Sequence; identical to SEQ ID NO: 61
  recombinant sequence